(19) United States
(12) Patent Application Publication
(10) Pub. No.: US 2025/0255716 A1
(43) Pub. Date: Aug. 14, 2025

(54) COMMISSURE ASSEMBLY FOR PROSTHETIC HEART VALVE

(71) Applicant: EDWARDS LIFESCIENCES CORPORATION, Irvine, CA (US)

(72) Inventors: Jeanette Jasmine Corona Kelly, Orange, CA (US); Brendan Michael Dalbow, Oceanside, CA (US); Elena Sherman, Pardes Hana (IL); Nikolai Gurovich, Hadera (IL); Socheata Iep, Santa Ana, CA (US)

(73) Assignee: EDWARDS LIFESCIENCES CORPORATION, Irvine, CA (US)

(21) Appl. No.: 19/169,186

(22) Filed: Apr. 3, 2025

Related U.S. Application Data
(63) Continuation of application No. PCT/US2023/033741, filed on Sep. 26, 2023.
(60) Provisional application No. 63/380,021, filed on Oct. 18, 2022.

(51) Int. Cl. A61F 2/24

(52) U.S. Cl. CPC A61F 2/2415; A61F 2/2418; A61F 2220/0075

(57) ABSTRACT

An implantable prosthetic device can include a radially expandable and compressible annular frame comprising an inflow end portion and an outflow end portion, and a plurality of leaflets arranged within the frame. Each leaflet can include one or more commissure tabs. The commissure tabs of two adjacent leaflets coupled to a commissure attachment member to form a commissure. The commissure can be secured to the frame via first and second sutures, each suture extending through a respective commissure tab, through the commissure attachment member, crossing over the other suture and being secured to the frame. The commissure can define a bending axis disposed radially inwardly of the frame such that a main body of each leaflet is prevented from contacting the frame when the valvular structure is in an open configuration.

### CROSS REFERENCE TO RELATED APPLICATION

This application is a continuation of PCT Patent Application No. PCT/US2023/033741 filed on Sep. 26, 2023, which claims the benefit of U.S. Provisional Patent Application No. 63/380,021, filed on Oct. 18, 2022, each which is incorporated by reference herein in its entirety.

### FIELD

The present disclosure relates to prosthetic heart valves, and to methods and assemblies for forming leaflet assemblies and attaching the leaflet assemblies to the frame of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Known valves can have valvular structures attached to the frame in such a manner that the outflow edges of each leaflet are spaced radially inward of the frame to prevent leaflet abrasion when the leaflets open under the flow of blood. In such valves, the effective outflow orifice (for example, as determined by the position of the leaflets in the open state), can be narrower than the inflow orifice, producing a relatively high pressure gradient across the prosthetic valve. High pressure gradients can lead to various clinical risks, such as cavitation.

### SUMMARY

Described herein are prosthetic heart valves, delivery apparatus, and methods for implanting prosthetic heart valves. The disclosed prosthetic heart valves, delivery apparatus, and methods can, for example, provide commissure configurations that maximize the effective outflow orifice while mitigating damage to the leaflets from contacting the frame. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, the prosthetic valve can comprise commissures configured to position a bending axis of the leaflet radially inwardly from the frame such that a main body of each leaflet is prevented from contacting the frame when the valvular structure is in an open configuration.

In some examples, the prosthetic valve can comprise a commissure having first and second sutures that couple the commissure to the frame, the first and second sutures crossing over one another at a location radially inward of the frame.

In some examples, the prosthetic valve can comprise a commissure attachment member.

In some examples, the prosthetic valve can comprise commissures each comprising a reinforcing member.

In some examples, the commissures can comprise third and fourth sutures, and each of the third and fourth sutures can extend through a respective reinforcing member, a respective commissure tab, and the commissure attachment member.

In some examples, the first and second sutures can each extend through a respective reinforcing member.

In some examples, each leaflet can comprise one or more commissure tabs. In some examples, each commissure tab includes an upper tab portion and a lower tab portion and the upper tab portion can be folded toward the inflow end of the prosthetic device to form first and second tab layers.

In some examples, an outer edge of the lower tab portion extends laterally past an outer edge of the upper tab portion.

In some examples, an outflow edge of the lower tab portion of each leaflet is disposed at a non-90 degree angle relative to a longitudinal axis of the leaflet.

In some examples, the width of the upper tab portion of the commissure tab has a width of 1 mm.

In some examples, a radially inner edge portion of each first tab layer wraps around a portion of the commissure attachment member such that the main body of the leaflet contacts the first tab layer when the valvular structure is in the open configuration.

In some examples, at least one of the first, second, third, and fourth sutures is secured to the frame using one or more whip stitches.

In some examples, each commissure tab comprises first and second alignment features wherein when an outer portion of the tab is folded toward the longitudinal axis of the leaflet, the first alignment feature aligns with the second alignment feature.

In some examples, the first and second side portions of the commissure attachment member have an elongated shape.

In some examples, an implantable prosthetic device comprises a radially expandable and compressible annular frame comprising an inflow end portion and an outflow end portion, a valvular structure comprising a plurality of leaflets arranged within the frame, each leaflet comprising a main body and one or more commissure tabs, and a commissure comprising a commissure attachment member and the commissure tabs of two adjacent leaflets coupled to the commissure attachment member. The commissure being secured to the frame via first and second sutures, each suture extending through a respective commissure tab, through the commissure attachment member, crossing over the other suture and being secured to the frame. The commissure can define a bending axis disposed radially inwardly of the frame such that the main body of each leaflet is prevented from contacting the frame when the valvular structure is in an open configuration.

In some examples, a method of assembling a valvular structure, comprises folding an integral upper tab portion of a first leaflet onto an integral lower tab portion of the first leaflet to form first and second tab layers, disposing a first reinforcing member on the second tab layer of the first leaflet, disposing the first and second tab layers on a first side portion of a commissure attachment member, and coupling the first reinforcing member, the first tab layer, the second tab layer, and the first side portion of the commissure attachment member to one another using one or more sutures. The method further comprises folding an integral upper tab portion of a second leaflet onto an integral lower tab portion of the first leaflet to form first and second tab layers, disposing a second reinforcing member on the second tab layer of the second leaflet, disposing the first and second tab layers on a second side portion of the commissure attachment member, and coupling the second reinforcing member, the first tab layer and the second tab layer of the second leaflet, and the second side portion of the commissure attachment member to one another using one or more sutures to form a commissure.

In some examples, an implantable prosthetic device, comprises a frame comprising an inflow end portion and an outflow end portion, the frame being radially collapsible to a collapsed configuration and radially expandable to an expanded configuration, a valvular structure comprising a plurality of leaflets arranged within the frame, each leaflet comprising a main body and a pair of opposing commissure tabs extending from opposite sides of the main body, each commissure tab having a lower tab portion and an upper tab portion, and a commissure attachment member comprising a main body portion and first and second side portions. The prosthetic device can further comprise a commissure comprising the commissure tabs of two adjacent leaflets coupled to the commissure attachment member, the upper tab portion of each tab being folded toward the inflow end of the frame to form first and second tab layers. A first suture can couple the first and second tab layers of a respective first leaflet to the first side portion of the commissure attachment member and a second suture can couple the first and second tab layers of a respective second leaflet to the second side portion. A third suture can extend through the first side portion, the first tab layer of the first leaflet, the main body portion of the commissure attachment member, and can be secured to the frame, and a fourth suture can extend through the second side portion, the first tab layer of the second leaflet, the main body portion of the commissure attachment member and can be secured to the frame. The third and fourth sutures can cross over one another adjacent the frame, and the commissure can define a bending axis disposed radially inwardly of the frame such that the main body of the leaflet is prevented from contacting the frame when the valvular structure is in an open configuration.

In some examples, an implantable prosthetic device, comprises a radially expandable and compressible annular frame comprising an inflow end portion and an outflow end portion, and a valvular structure comprising a plurality of leaflets arranged within the frame. Each leaflet can comprise a main body and one or more commissure tabs, an outer portion of each commissure tab being folded toward a longitudinal axis of the leaflet to form first and second tab layers. The prosthetic device can comprise a commissure comprising a commissure attachment member and the commissure tabs of two adjacent leaflets coupled to the commissure attachment member. The commissure can be secured to the frame via first and second sutures, each suture extending through the first tab layer, the second tab layer, the commissure attachment member, crossing over the other suture and being secured to the frame. The commissure can define a bending axis disposed radially inwardly of the frame such that the main body of each leaflet is prevented from contacting the frame when the valvular structure is in an open configuration.

In some examples, an implantable prosthetic device, comprises a radially expandable and compressible frame comprising an inflow end portion and an outflow end portion, and a valvular structure comprising a plurality of leaflets arranged within the frame. Each leaflet can comprise a main body and one or more commissure tabs, each commissure tab comprising a first alignment feature on a laterally outer tab portion and a second alignment feature on a laterally inner tab portion, the laterally outer portion of each commissure tab being folded over the laterally inner portion such that the first alignment feature is disposed on the second alignment feature to form first and second tab layers. The prosthetic device can further include a commissure comprising a commissure attachment member and the commissure tabs of a first leaflet and an adjacent second leaflet coupled to the commissure attachment member, the commissure comprising a first suture extending through the first and second tab layers of the first leaflet and a second suture extending through the first and second tab layers of the second leaflet. The commissure can be secured to the frame via third and fourth sutures, the third suture extending through the first and second tab layers of the first leaflet, the commissure attachment member, crossing over the fourth suture, and being secured to the frame at a first location, and the fourth suture extending through the first and second tab layers of the second leaflet, the commissure attachment member, crossing over the third suture, and being secured to the frame at a second location. The commissure can define a bending axis disposed radially inwardly of the frame such that the main body of each leaflet is prevented from contacting the frame when the valvular structure is in an open configuration.

In some examples, a method of assembling a valvular structure, comprises disposing a first reinforcing member on a laterally inner tab portion of a commissure tab of a first leaflet such that the first reinforcing member is aligned with a first alignment feature of the laterally inner tab portion, folding a laterally outer tab portion laterally over the first reinforcing member such that a second alignment feature disposed on the outer tab portion of the commissure tab of the first leaflet aligns with the first alignment feature, and such that the commissure tab forms first and second tab layers, coupling the first and second tab layers and the first reinforcing member using one or more first sutures, and disposing the first and second tab layers and the first reinforcing member on a first side portion of a commissure attachment member. The method can further comprise disposing a second reinforcing member on a laterally inner tab portion of a commissure tab of a second leaflet such that the second reinforcing member is aligned with a first alignment feature of the laterally inner tab portion, folding a laterally outer tab portion of the second leaflet laterally over the second reinforcing member such that a second alignment feature disposed on the outer tab portion of the commissure tab of the second leaflet aligns with the first alignment feature, and such that the commissure tab forms first and second tab layers, coupling the first and second tab layers and the second reinforcing member using one or more second sutures, and disposing the first and second tab layers and the second reinforcing member on a second side portion of the commissure attachment member to form a commissure. The method can include coupling the commissure to a radially expandable and compressible annular frame via one or more third sutures extending diagonally through the first and second tab layers of the first leaflet and one or more fourth sutures extending diagonally through the first and second tab layers of the second leaflet, the third and fourth sutures crossing over one another at a location radially inside the frame.

In some examples, an implantable prosthetic device, comprises a radially expandable and compressible annular frame comprising an inflow end portion and an outflow end portion, and a valvular structure comprising a plurality of leaflets arranged within the frame. Each leaflet comprises a main body and one or more commissure tabs, each commissure tab having an upper tab portion and a lower tab portion, the upper tab portion being folded toward an inflow end portion of the leaflet to form first and second tab layers. The prosthetic device can further comprise one or more reinforcing members each disposed on a respective second tab layer of a commissure tab, and a commissure comprising a commissure attachment member and the commissure tabs of two adjacent leaflets coupled to the commissure attachment member. The commissure can be secured to the frame via first and second sutures, each suture extending radially through a respective reinforcing member and the first and second tab layers of the two adjacent leaflets and being secured to the frame. The commissure can define a bending axis disposed radially inwardly of the frame such that the main body of each leaflet is prevented from contacting the frame when the valvular structure is in an open configuration.

In some examples, a method of assembling a valvular structure, comprises folding an integral upper tab portion of a first leaflet onto an integral lower tab portion of the first leaflet to form first and second tab layers, disposing a first reinforcing member on the second tab layer of the first leaflet, disposing the first and second tab layers on a first side portion of a commissure attachment member, and coupling the first reinforcing member, the first tab layer, the second tab layer, and the first side portion of the commissure attachment member to one another using one or more sutures. The method can further comprise folding an integral upper tab portion of a second leaflet onto an integral lower tab portion of the second leaflet to form first and second tab layers, disposing a second reinforcing member on the second tab layer of the second leaflet, disposing the first and second tab layers on a second side portion of the commissure attachment member, and coupling the second reinforcing member, the first tab layer and the second tab layer of the second leaflet, and the second side portion of the commissure attachment member to one another using one or more sutures to form a commissure. The commissure can be coupled to a radially expandable and compressible annular frame via one or more third sutures extending diagonally through the first reinforcing member and the first and second tab layers of the first leaflet and one or more fourth sutures extending diagonally through the second reinforcing member and the first and second tab layers of the second leaflet, the third and fourth sutures crossing over one another at a location radially inside the frame.

In some examples, an implantable prosthetic device, comprises a radially expandable and compressible annular frame comprising an inflow end portion and an outflow end portion, a valvular structure comprising a plurality of leaflets arranged within the frame, each leaflet comprising a main body and one or more commissure tabs, and a commissure attachment member comprising a main body, a first side portion, and a second side portion. The device can further comprise a commissure comprising the commissure attachment member and the commissure tabs of first and second adjacent leaflets coupled to the commissure attachment member, the first side portion of the commissure attachment member being folded over a radially outer edge of a first commissure tab of the first leaflet to form first and second commissure member layers, and the second side portion of the commissure attachment member being folded over a radially outer edge of a second commissure tab of the second leaflet to form first and second commissure member layers. The commissure can be secured to the frame via first and second sutures, the first suture extending radially through the first and second commissure member layers of the first side portion and the first commissure tab, the second suture extending radially through the first and second commissure member layers of the second side portion and the second commissure tab. The commissure can be further secured to the frame via a third suture extending diagonally through the commissure in a first direction and a fourth suture extending diagonally through the commissure in a second direction such that the third and fourth sutures cross over one another in an X shape, the third and fourth sutures extending around respective struts of the frame in a plurality of whip stitches.

In some examples, a method of assembling a valvular structure, comprises folding a first side portion of a commissure attachment member laterally toward a main body of the commissure attachment member to form a first commissure member layer and a second commissure member layer, and folding a second side portion of the commissure attachment member laterally toward the main body to form a first commissure member layer and a second commissure member layer. The method can further comprise disposing a radially outer edge of a first commissure tab of a first leaflet between the first and second commissure member layers of the first side portion, and disposing a radially outer edge of a second commissure tab of a second leaflet between the first and second commissure member layers of the second side portion. The method can further comprise disposing a first reinforcing member on the first commissure member layer of the first side portion and a second reinforcing member on the first commissure member layer of the second side portion, coupling the first reinforcing member, the first and second commissure member layers of the first side portion, and the first commissure tab one another using one or more first sutures, and coupling the second reinforcing member, the first and second commissure member layers of the second side portion, and the second commissure tab one another using one or more second sutures to form a commissure. The method can further comprise coupling the commissure to a radially expandable and compressible annular frame via the first and second sutures, sewing one or more third sutures diagonally through the commissure in a first direction and one or more fourth sutures diagonally through the commissure in a second direction such that the third and fourth sutures cross over one another in an X shape, and securing the one or more third and fourth sutures to selected struts of the frame using a whip-stitch pattern.

In some examples, a method comprises sterilizing any of the above described devices.

The above method(s) can be performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic heart valve, according to one example.

FIG. 1B is a perspective view of the prosthetic valve of FIG. 1A with the components on the outside of the frame shown in transparent lines for purpose of illustration.

FIG. 2 is a side view of an example of a delivery apparatus configured to deliver and implant a radially expandable prosthetic heart valve at an implantation site.

FIG. 3 is a plan view of an example of a leaflet of a prosthetic heart valve.

FIG. 4 is a plan view of an example of a commissure attachment member.

FIG. 5 is a cross-sectional view of a commissure coupled to the frame of a prosthetic heart valve.

FIG. 6 is a cross-sectional view of a commissure coupled to the frame of a prosthetic heart valve.

FIG. 7 is a cross-sectional view of a commissure coupled to the frame of a prosthetic heart valve.

FIG. 8 is a top plan view of a portion of a prosthetic heart valve including the commissure of FIG. 7.

FIG. 9 is a cross-sectional view of a commissure coupled to the frame of a prosthetic heart valve.

FIGS. 10-11 are plan views of an example of a leaflet of a prosthetic heart valve.

FIG. 12 is a cross-sectional view of a commissure of a prosthetic heart valve including the leaflet of FIGS. 10-11 coupled to the frame of a prosthetic heart valve.

FIG. 13 is a top plan view of a prosthetic heart valve including the commissures of FIG. 12.

FIG. 14 is a top plan view of one of the commissures of the prosthetic valve of FIG. 13.

FIG. 15 is a perspective view of the commissure of FIG. 12 coupled to the frame of a prosthetic valve.

FIG. 16A is a plan view of an example of a leaflet for a prosthetic valve.

FIG. 16B is a plan view of an example of a commissure attachment member.

FIGS. 17-20 are cross-sectional views illustrating steps for assembling a commissure and coupling the commissure to the frame of a prosthetic valve.

FIGS. 21-23 are perspective views of the commissure of FIG. 20 coupled to the frame of a prosthetic valve.

FIG. 24 is a plan view of an example of a leaflet for a prosthetic valve.

FIG. 25 is a plan view of an examples of a commissure attachment member for a prosthetic valve.

FIGS. 26-29 are cross-sectional views illustrating steps for assembling a commissure and coupling the commissure to the frame of a prosthetic valve.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

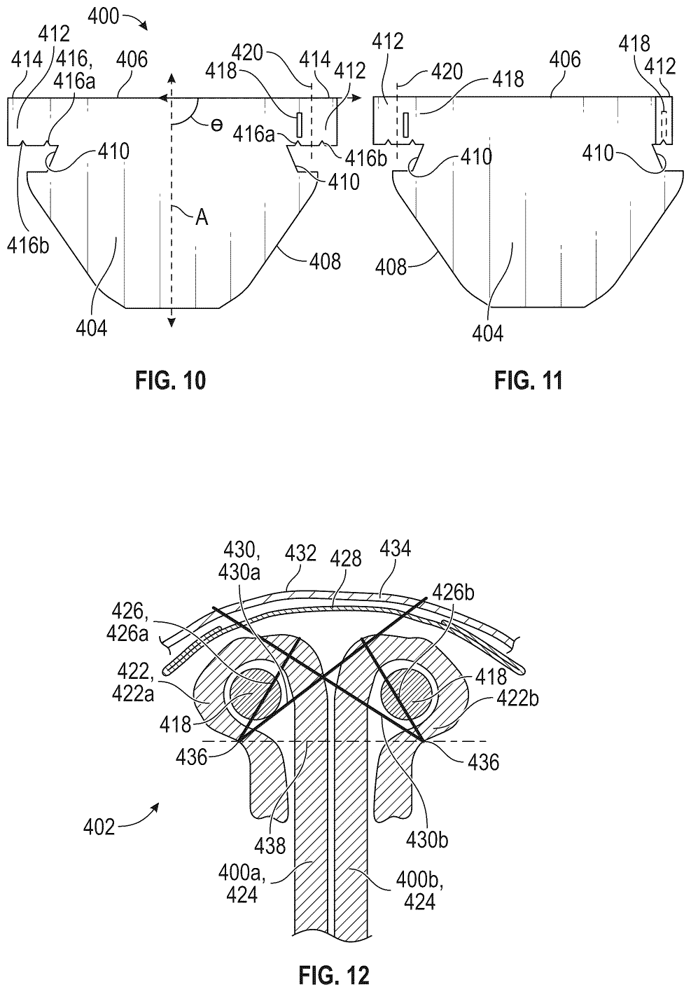

All features described herein are independent of one another and, except where structurally impossible, can be used in combination with any other feature described herein. For example, a commissure attachment member 604 as shown in FIG. 25 can be used in combination with any of the commissures 338, 402, or 502 described herein. In some examples, any of the commissures 338, 402, 502, 602 described herein can be used with prosthetic valve 150. In an example, leaflet tabs 412 or alignment features 416 as shown in FIG. 10 can be used in combination with the commissures 338, 502, or 602. In some examples, the whip-stitch securing methods shown in FIG. 20 and FIG. 29 can be used with any of the disclosed commissures.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Examples of the Disclosed Technology

Described herein are examples of prosthetic heart valves, leaflet assemblies for prosthetic heart valves, connecting skirts, commissure attachment members, and methods for assembling leaflets assemblies and valvular structures to a frame of the prosthetic heart valve. The prosthetic heart valves can comprise a frame and a valvular structure attached to the frame, the valvular structure comprising a plurality of leaflets. In some examples, the valvular structure can be secured to the frame via one or more commissure attachment members.

Prosthetic valves disclosed herein can be radially compressible and expandable between a radially compressed configuration and a radially expanded configuration. Thus, the prosthetic valves can be crimped on or retained by an implant delivery apparatus in the radially compressed configuration during delivery, and then expanded to the radially expanded configuration once the prosthetic valve reaches the implantation site. It is understood that the prosthetic valves disclosed herein may be used with a variety of implant delivery apparatuses and can be implanted via various delivery procedures, examples of which will be discussed in more detail later. In some examples, the prosthetic valve can be deployed from the delivery apparatus at the implantation site (for example, a native valve of a heart) via inflating an inflatable balloon of the delivery apparatus. When radially compressed onto the inflatable balloon of the delivery apparatus, inner surfaces of the frame of the prosthetic valve can face the balloon.

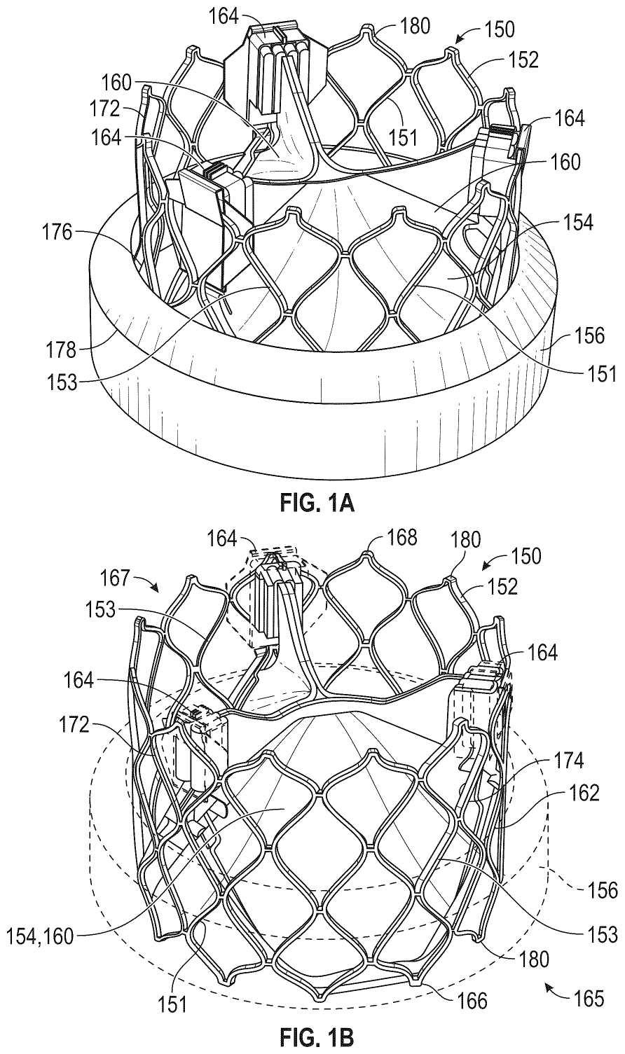

FIG. 1A shows an exemplary prosthetic valve 150, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

FIG. 1A is a perspective view of a prosthetic heart valve (for example, prosthetic valve) 150, according to one example. The illustrated prosthetic valve is adapted to be implanted in the native aortic annulus, although in some examples it can be adapted to be implanted in the other native annuluses of the heart (for example, the pulmonary, mitral, and tricuspid valves). The prosthetic valve can also be adapted to be implanted in other tubular organs or passageways in the body.

The prosthetic valve 150 can have three main components: a stent or frame, 152, a valvular structure 154, and a sealing member 156. FIG. 1B is a perspective view of the prosthetic valve 150 with the components on the outside of the frame 152 (including the sealing member 156) shown in transparent lines for purposes of illustration. The frame 152 can comprise a plurality of struts 153 arranged to define a plurality of diamond-shaped cells 151. The cells 151 can be arranged in circumferentially extending rows. The prosthetic valve 150 can have an inflow end portion 165 including an inflow end 166 and an outflow end portion 167 including an outflow end 168.

The valvular structure 154 can comprise three leaflets 160, collectively forming a leaflet structure, which can be arranged to collapse in a tricuspid arrangement, although in some examples there can be greater or fewer number of leaflets (for example, one or more leaflets 160). In some examples, the leaflets 160 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

Each leaflet 160 can be coupled to the frame 152 along its inflow edge 162 (FIG. 1B; the lower edge in the figures; also referred to as “cusp edges”) and at commissures 164 of the valvular structure 154 where adjacent portions (for example, commissure tabs) of two leaflets are connected to each other. In some examples, the commissures 164 can comprise an attachment member (for example, comprising fabric, flexible polymer, or the like) arranged across a cell 151 of the frame 152, the cell 151 formed by struts 153 of the frame. The cell to which a commissure is attached can also be referred to as a ‘commissure cell.’ The attachment member can be secured to the struts 153 of the frame forming the commissure cell via stitching or sutures. Further, the adjacent portions of the two leaflets can be connected to the attachment member to form the commissure 164.

In some examples, a reinforcing element or connecting skirt, such as a fabric strip, can be connected directly to the cusp edges 162 of the leaflets and to the struts of the frame to couple the cusp edges of the leaflets to the frame. In some examples, the cusp edges 162 can be coupled directly to the frame 152.

The frame 152 can be made of any of various suitable plastically-expandable materials (for example, stainless steel, etc.) or self-expanding materials (for example, nickel titanium alloy (NiTi), such as nitinol), as known in the art. When constructed of a plastically-expandable material, the frame 152 (and thus the prosthetic valve 150) can be crimped to a radially collapsed configuration on a delivery apparatus (for example, catheter) and then expanded inside a patient by an inflatable balloon or equivalent expansion mechanism. When constructed of a self-expandable material, the frame 152 (and thus the prosthetic valve 150) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Suitable plastically-expandable materials that can be used to form the frames disclosed herein (for example, the frame 152) include, metal alloys, polymers, or combinations thereof. Example metal alloys can comprise one or more of the following: nickel, cobalt, chromium, molybdenum, titanium, or other biocompatible metal. In some examples, the frame 152 can comprise stainless steel. In some examples, the frame 152 can comprise cobalt-chromium. In some examples, the frame 152 can comprise nickel-cobalt-chromium. In some examples, the frame 152 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The frame 152 in the illustrated example comprises a plurality of circumferentially extending rows of angled struts 172 defining rows of open cells, or openings, 174 of the frame. The frame 152 can have a cylindrical or substantially cylindrical shape having a constant diameter from the inflow end 166 to the outflow end 168 of the frame 152 as shown, or the frame 152 can vary in diameter along the height of the frame, as disclosed in U.S. Pat. No. 9,155,619, which is incorporated herein by reference. The frame 152, at each of the inflow end 166 and the outflow end 168, may comprise a plurality of apices 180 spaced apart from one another around a circumference of the frame 152.

The sealing member 156 in the illustrated example is mounted on the outside of the frame 152 and functions to create a seal against the surrounding tissue (for example, the native leaflets and/or native annulus) to prevent or at least minimize paravalvular leakage. The sealing member 156 can comprise an inner layer 176 (which can be in contact with the outer surface of the frame 152) and an outer layer 178. The sealing member 156 can be connected to the frame 152 using suitable techniques or mechanisms. For example, the sealing member 156 can be sutured to the frame 152 via sutures that can extend around the struts 172 and through the inner layer 176. In alternative examples, the inner layer 176 can be mounted on the inner surface of the frame 152, while the outer layer 178 is on the outside of the frame 152. In some examples, the sealing member 156 can comprise only a single layer disposed on the radially outer surface of the frame 152.

Additional details regarding the prosthetic valve 150 and its various components are described in U.S. Pat. No. 11,096,781, which is incorporated herein by reference in its entirety.

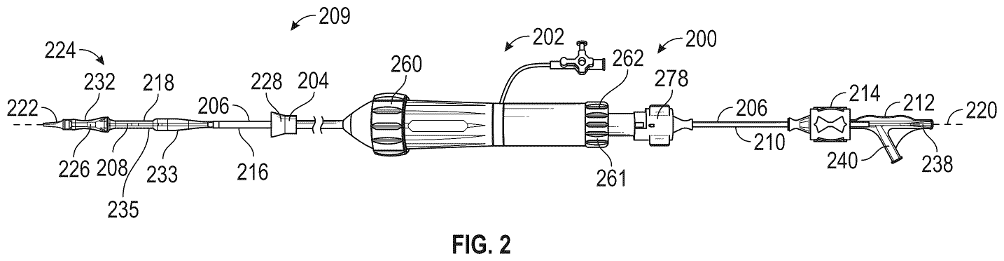

FIG. 2 shows a delivery apparatus 200, according to an example, that can be used to implant an expandable prosthetic heart valve (for example, prosthetic valve 150 of FIGS. 1A and 1B), or another type of expandable prosthetic medical device (such as a stent). In some examples, the delivery apparatus 200 is specifically adapted for use in introducing a prosthetic valve into a heart.

The delivery apparatus 200 in the illustrated example of FIG. 2 is a balloon catheter comprising a handle 202 and a steerable, outer shaft 204 extending distally from the handle 202. The delivery apparatus 200 can further comprise an intermediate shaft 206 (which can also be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. In some examples, a rotatable knob 214 can be mounted on the proximal end portion 210 and can be configured to rotate the intermediate shaft 206 around the central longitudinal axis 220 of the delivery apparatus 200 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion 216 that extends distally beyond a distal end of the outer shaft 204 when a distal end of the outer shaft 204 is positioned away from an inflatable balloon 218 of the delivery apparatus. A distal end portion of the inner shaft 208 can extend distally beyond the distal end portion 216 of the intermediate shaft 206.

Referring still to FIG. 4, the balloon 218 can be coupled to the distal end portion 216 of the intermediate shaft 206. For example, in some examples, a proximal end portion of the balloon 218 can be coupled to and/or around a distal end of the intermediate shaft 206. The balloon 218 can comprise a distal end portion (or section) 232, a proximal end portion (or section) 233, and an intermediate portion (or section) 235, the intermediate portion 235 disposed between the distal end portion 232 and the proximal end portion 233.

In some examples, a distal end of the distal end portion 232 of the balloon 218 can be coupled to a distal end of the delivery apparatus 200, such as to a nose cone 222, or to an alternate component at the distal end of the delivery apparatus 200 (for example, a distal shoulder). In some examples, the intermediate portion 235 of the balloon 218 can overlay a valve mounting portion 224 of a distal end portion 209 of the delivery apparatus 200, the distal end portion 232 can overly a distal shoulder 226 of the delivery apparatus 200, and the proximal end portion 233 can surround a portion of the inner shaft 208. The valve mounting portion 224 and the intermediate portion 235 of the balloon 218 can be configured to receive a prosthetic heart valve (such as prosthetic valve 150 described previously) in a radially compressed state.

In some examples, rotation of the intermediate shaft 206 can result in rotation of the balloon 218 and the prosthetic valve mounted thereon for rotational positioning of the prosthetic valve relative to the native anatomy at the target implantation site.

The balloon shoulder assembly is configured to maintain the prosthetic heart valve or other medical device at a fixed position on the balloon 218 during delivery through the patient's vasculature. Further details of the balloon shoulder assembly can be found, for example, in International Publication No. WO2022159427A1, which is incorporated by reference herein in its entirety.

As shown in FIG. 4, the outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when a prosthetic valve is mounted in the radially compressed state on the valve mounting portion 224 and during delivery of the prosthetic valve to the target implantation site. As such, the distal tip portion 228 can be configured to resist movement of the prosthetic valve relative to the balloon 218 proximally, in the axial direction, relative to the balloon 218, when the distal tip portion 228 is arranged adjacent to a proximal side of the valve mounting portion 224.

In some examples, the nose cone 222 can be disposed distal to and be coupled to the distal shoulder 226. In some examples, the nose cone 222 can be coupled to the distal end portion of the inner shaft 208.

In some examples, an annular space can be defined between an outer surface of the inner shaft 208 and an inner surface of the intermediate shaft 206. In some examples, the annular space can be referred to as an inner lumen of the intermediate shaft 206. In some examples, the annular space can be configured to receive fluid from a fluid source via the second port 240 of the adaptor 212 (for example, the annular space is in fluid communication with the second port 240 of the adaptor 212). The annular space can be fluidly coupled to a fluid passageway formed between the outer surface of the distal end portion of the inner shaft 208 and an inner surface of the balloon 218. As such, fluid from the fluid source can flow to the balloon 218 to inflate the balloon 218 and radially expand and deploy the prosthetic valve.

An inner lumen of the inner shaft 208 can be configured to receive a guidewire therethrough, for navigating the distal end portion 209 of the delivery apparatus 200 to the target implantation site. As introduced above, the first port 238 of the adaptor 212 can be coupled to the inner lumen and configured to receive the guidewire. For example, the distal end portion 209 of the delivery apparatus 200 can be advanced over the guidewire, to the target implantation site.

As shown in FIG. 4, the handle 202 can include a steering mechanism configured to adjust the curvature of the distal end portion 209 of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion 209 of the delivery apparatus 200. Further details on steering or flex mechanisms for the delivery apparatus can be found in U.S. Pat. No. 9,339,384, which is incorporated by reference herein in its entirety.

The handle 202 can include one or more additional adjustment mechanisms. For example, in some examples, the handle 202 can include an adjustment mechanism 261 including an adjustment member, such as the illustrated rotatable knob 262. The adjustment mechanism 261 can be configured to adjust the axial position of the intermediate shaft 206 relative to the outer shaft 204. In some examples, the handle 202 can further include a locking mechanism, which can include a rotatable knob 278, the locking mechanism configured to retain (for example, lock) the position of the intermediate shaft 206 relative to the handle 202 and allow for fine positioning of the prosthetic valve at the implantation site.

During typical valve operation, the leaflets transition between a closed state in diastole, with their outflow edges coapting against each other, and an open state allowing blood to flow through the prosthetic valve, such as prosthetic valve 150 described previously. The outflow orifice through which the blood can flow determines the pressure gradient across the valve. Known valves can have valvular structures attached to the frame in such a manner that the outflow edges of each leaflet are spaced radially inward of the frame to prevent leaflet abrasion when the leaflets open under the flow of blood. In such valves, the effective outflow orifice (for example, as determined by the position of the leaflets), also referred to as the geometric orifice area (GOA), can be narrower than the inflow orifice, producing a relatively high pressure gradient across the prosthetic valve. The term “GOA,” as used herein, is defined as the open space through which blood can flow when the valvular structure is in the open configuration. The increased pressure gradient can lead to prosthesis-patient-mismatch (PPM) which may lead to complications. Accordingly, it is preferable to provide a large outflow orifice during systole of the aortic valve to prevent elevated pressure gradients.

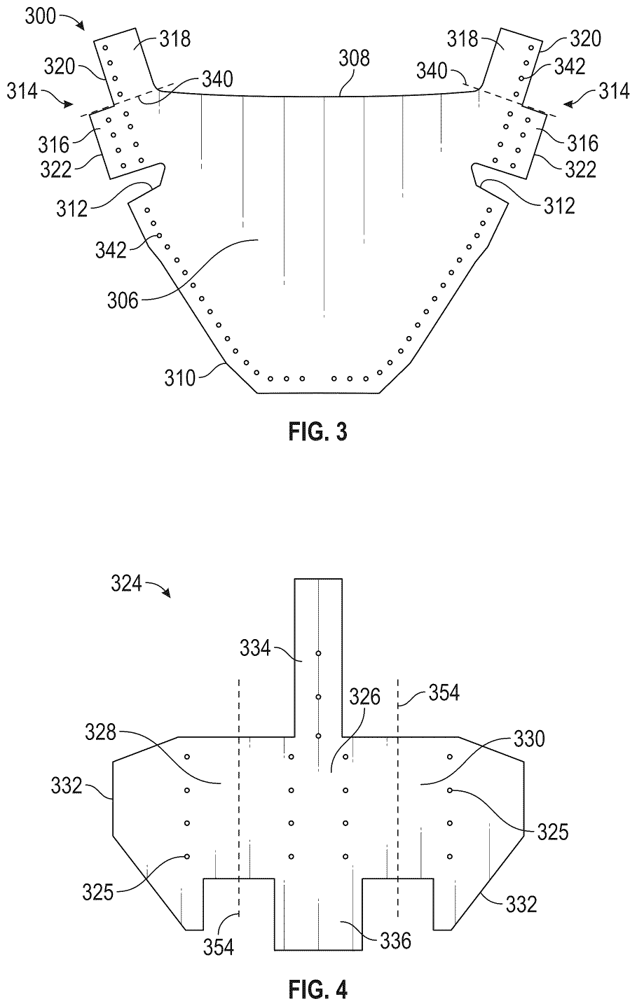

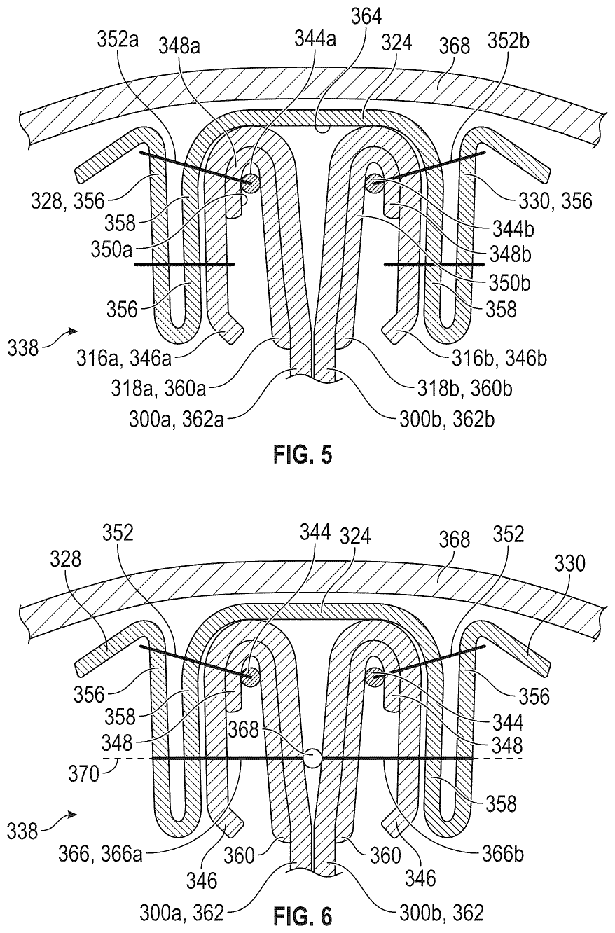

FIGS. 3-9 illustrate an example of a leaflet 300, a commissure attachment member 324, and a method for forming a commissure 304 from two leaflets 300 and the commissure attachment member 324. As shown in FIG. 3, the leaflet 300 comprises a main body 306, an upper edge portion 308 (also referred to as the coaptation edge), a lower edge portion 310 (also referred to as the cusp edge or scallop edge) terminating at openings/recesses/gaps 312 that extend laterally into the main body of the leaflet, and tabs 314 (also referred to as commissure tabs), which are spaced from the lower edge portion 310 by the gaps 312. In some examples, the tabs can be formed integrally with the main body of the leaflet. The cusp edge 310 can be mounted to the frame of a prosthetic heart valve, for example, prosthetic valve 150 described earlier, for example using a connecting skirt. In some examples, during assembly to the frame, the lower cusp edge portion 310 can become folded toward the frame. As described further below and shown in FIGS. 5-9, the tabs 314 can be folded over on themselves and coupled to the commissure attachment member 324 to form commissures.

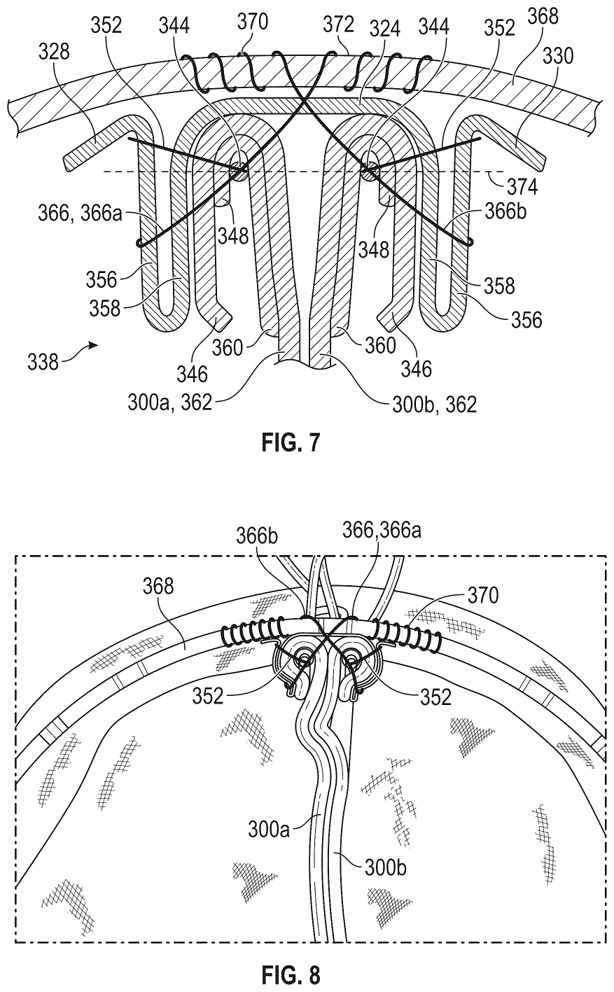

Each tab 314 can include a lower tab portion 316 and an upper tab portion 318. As shown in FIG. 3, the upper tab portion 318 can have an outer edge 320 that is inset from an outer edge 322 of the lower tab portion 316, forming a stepped configuration. Such a configuration removes excess material from the commissure (for example, by allowing for fewer folded layers) while creating additional support (for example, multiple layers) at the bending axis (see for example, FIG. 7). In some examples, such as the illustrated example, the tabs 314 and the cusp edge portion 310 can comprise one or more preformed openings or apertures 342 configured to facilitate sewing one or more components of the prosthetic valve to one another. In some examples, the openings may not be preformed in the leaflet 300. In alternate examples, the leaflets 300 of the valvular structure can be configured differently than the leaflet shown in FIG. 3, such as having differently sized or shaped upper or lower tab portions 316, 318 or differently sized or shaped gaps 312.

An example of a commissure attachment member 324 in a flat and unfolded state (for example, prior to folding and commissure formation) is shown in FIG. 4. It should be noted that, in alternative examples, the attachment member 324 can have different configurations than shown in FIG. 4, such as not including the upper and/or lower extensions 334, 336, or having a different overall shape (for example a rectangular shape, a hexagonal shape, or the like).

Referring to FIG. 4, the commissure attachment member 324 can have a main body portion 326, a first side portion 328 and an opposing second side portion 330 projecting laterally from the main body portion 326. As shown, the outer peripheral edges 332 of the side portions 328, 330 can be shaped to correspond to one half of a diamond-shaped cell of the frame (for example, cell 151 of frame 152) to facilitate mounting of the commissure attachment member 324 to the struts 153 of the frame 152. The commissure attachment member 324 can further comprise an upper extension 334 and a lower extension 336 that extend from the main body 326. In some examples, the upper and/or lower extension 334, 336 can be optional. In some examples, the upper extension can comprise a radiopaque marker used to indicate the location of the commissure.

In some examples, such as the illustrated example, the commissure attachment member 324 can comprise one or more preformed openings 325 configured to facilitate sewing one or more components of the prosthetic valve to one another. In some examples, the openings may not be preformed in the commissure attachment member 324. In some examples, the commissure attachment member 324 can comprise a fabric, a flexible polymeric material, or a combination thereof. For example, the commissure attachment member can comprise polyethylene terephthalate (PET).

Referring to FIGS. 5-8, to form a commissure 338, the upper tab portion 318a of a first leaflet 300a is folded along fold line 340 (FIG. 3) against the lower tab portion 316a such that the apertures 342 of the upper tab portion 318a align with an inner line of apertures 342 of the lower tab portion 316a. This creates a first tab layer 346a (previously the lower tab portion 316a) and a second layer 348a (previously the upper tab portion 318a).

Referring to FIG. 5, a reinforcement member 344a, such as a suture or strip of fabric (for example, an ethibond suture or strip of PET), can be secured to the inner surface 350a of the second tab layer 348a such as with a first suture or stitch line 352a. The commissure attachment member 324 can be folded as shown (for example, along vertical fold lines 354 shown in FIG. 4) so that the side portions 328, 330 each comprise first and second layers 356, 358 of material. In the illustrated example, the first layer 356 is the outer layer and the second layer 358 is the inner layer.

The folded tab layers 346a, 348a can be secured to a side portion, such as the first side portion 328 of the commissure attachment member 324. The folded tab layers 346a, 348a can additionally be folded along a vertical fold line at the reinforcing member 344 into a U-shape forming a first layer 346, a second layer 348, a third layer 360, and a fourth layer 362 from each commissure tab 314, as depicted in FIGS. 5-7. The reinforcing member 344a can be positioned between the second layer 348a and the third layer 360a, as shown. The reinforcing member 344 can be, for example, a suture (for example, an ethibond suture), a strip of fabric (for example, PET), a braided cloth, a metallic braid, and/or a metal strip.

The commissure tab 314b of a second leaflet 300b can be folded in the same manner and placed against the folded commissure tab 314a of the first leaflet 300a within the commissure attachment member 324, for example, positioned against the radially inward facing surface 364. The side portions 328, 330 can be placed against respective first layers 346a, 346b of the leaflets 300a, 300b.

As shown in FIG. 5, the first suture or stitch line 352 can extend diagonally to secure the reinforcing member 344, the first and second layers 346, 348, and the first and second layers 356, 358 of a respective side portion (for example, side portion 328 or 330) of the commissure attachment member 324 to one another. The stitch lines can comprise a plurality of stitches of a suture (for example, in-and-out stitches).

In some examples, such as shown in FIG. 6, the commissure 338 can comprise a secondary stitch line 366 positioned radially inwardly of the first suture line 352. In some examples, such as shown in FIG. 6, the second stitch line 366 can be sewn laterally to couple the first and second layers 356, 358 of the commissure attachment member 324 to the first layer 346, the third layer 360, and the fourth layer 362 of the leaflet tab 314. The second stitch line 366a of a first leaflet 300a can be coupled to a respective second stitch line 366b of the second leaflet 300b via, for example, a knot 369, or other means of fastening. In such examples, the second stitch lines 366 define a bending axis 371 around which the leaflets 300 bend upon opening (for example, during systole of the aortic valve).

In some examples, as shown in FIG. 7-8, the commissure 338 can comprise a secondary stitch line 366 sewn such that it extends through the first and second layers 356, 358 of the commissure attachment member 324 and at least the first tab layer 346 and is then routed through the first stitch line 352 and the reinforcing member 344. This can advantageously reduce the width of the tab. The secondary stitch line 366a of the first leaflet 300a can cross over the secondary stitch line 366b of the second leaflet 300b. Such a configuration advantageously helps retain the commissure 338 in a substantially perpendicular orientation relative to the frame 368 and centered within a cell (for example, a commissure cell) of the frame. Frame 368 can be similar to frame 152 described previously.

The second stitch lines 366 can extend through the frame 368 and can be secured to the frame using one or more whip-stitches extending around selected struts of the frame 368. FIG. 7 illustrates the commissure configuration prior to tightening the secondary stitch lines 366 against the frame. FIG. 8 illustrates the commissure configuration once the secondary stitch lines 366 have been tightened against the frame. Such a configuration positions the bending axis 374 through the first stitch line 352. Such a configuration advantageously positions the bending axis 374 relatively closer to the frame 368 (for example, when compared to the example shown in FIG. 6) while still forming the bending axis 374 radially inwardly relative to the frame 368 to prevent the leaflets 300 from hitting against the frame.

The configuration shown in FIG. 7 advantageously prevents or mitigates the leaflets 300 from hitting the frame 368 when in the open state, which improves the long-term durability of the leaflets 300 by preventing or mitigating abrasion and/or damage. Such a configuration also advantageously allows the second stitch line 366 to provide added support for the tabs 314. The configuration of commissure 338 creates radially shorter tabs 314, thus allowing the leaflets 300 to open further, helping to maximize the effective outflow orifice (for example, as determined by the position of the leaflets), also referred to as the geometric orifice area (GOA), at the outflow end portion of the prosthetic valve, for example, prosthetic valve 150 described previously. The GOA of the outflow orifice can be sized to provide a selected pressure gradient across the prosthetic valve.

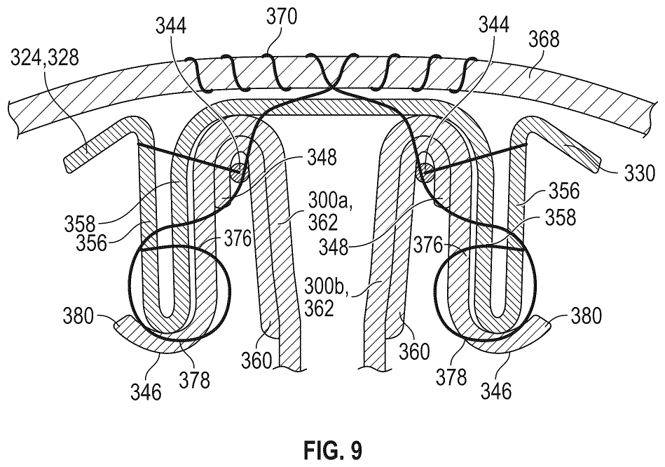

Referring now to FIG. 9, in some examples, the secondary stitch line 366 can be routed such that it extends through the first and second layers 356, 358 of the commissure attachment member 324 toward the tab 314, through the first tab layer 346 at a first location 376, and then through the first tab layer 346 at a second location 378 spaced radially inwardly from the first location 376. This allows a radially inner edge portion 380 of each respective first tab layer 346 to wrap around a respective side portion 328, 330 of the commissure attachment member 324 creating a softer/smoother folded portion against which the main body/working portion of the leaflet 300 can contact. Such a configuration can prevent or mitigate damaging and/or abrasive contact between the working portion of the leaflet (for example, the main body 306) and the edge portions of the other commissure components, such as commissure attachment member 324 and/or tabs 314.

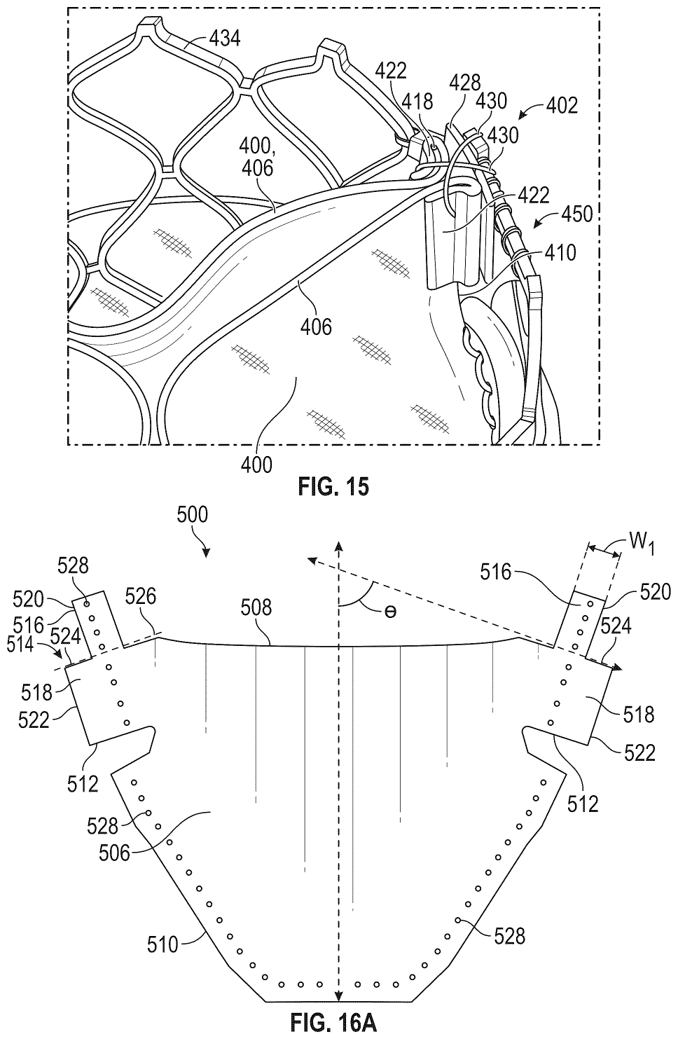

FIGS. 10-15 illustrate an example of a leaflet 400 and a method for forming a commissure 402 from two leaflets (for example, leaflets 400a and 400b), the commissure 402 being configured to help maximize the effective outflow orifice/GOA at the outflow end of a prosthetic valve, such as valve 150 described previously.

Referring to FIGS. 10-11, the leaflet 400 can be similar to leaflet 300 described previously, except that leaflet 400 does not include upper tab portions. As shown in FIG. 10, the leaflet can comprise a main body 404, an upper edge portion 406 (also referred to as the coaptation edge or free edge), a lower edge portion 408 (also referred to as the cusp edge or scallop edge) which terminates at openings/recesses/gaps 410 that extend laterally into the main body 404 of the leaflet, and laterally projecting integral tabs 412 (also referred to as commissure tabs), which are spaced from the lower edge portion by the gaps 410. In some examples, the tabs 412 can be formed integrally with the main body of the leaflet. The tabs 412 can extend from the main body 404 such that an upper or outflow edge 414 of each tab 412 is positioned at an angle Θ relative to a longitudinal axis A of the leaflet 400. In the illustrated example, the tabs 412 can extend from the main body 404 such that the angle Θ is a 90 degree angle. In some examples, the angle Θ can be any of various angles, for example, corresponding to (for example, being substantially parallel to) the draft angle of the frame of the prosthetic valve.

Each commissure tab 412 can comprise one or more alignment features/cutouts/recesses/notches 416. In the illustrated example, each tab 412 comprises a first alignment feature 416a disposed on a laterally inner portion of the tab 412 and a second alignment feature 416b disposed on a laterally outer portion of the tab 412. However, in some examples each tab 412 can comprise a greater or fewer number of alignment features. In the illustrated example, the alignment features 416 can be notches having a triangular shape, however in some examples the alignment features can have any shape including but not limited to square, rectangular, semi-circular, linear, etc. In the illustrated example, the alignment features 416 can be disposed on an inflow edge portion of the tab 412 and can extend toward the outflow edge portion 414. However, in some examples, the alignment features 416 can be disposed on any portion of the tabs, for example, the outflow edge portion 414.

To form a commissure 402, a reinforcing member 418 can be placed above the first notch 416a, such as shown in FIG. 10. The reinforcing member 418 can be, for example, a suture (for example, an ethibond suture), a strip of fabric (for example, PET), a braided cloth, a metallic braid, and/or a metal strip. The tab 412 can be folded along a vertical fold line 420 such that the second alignment feature 416b aligns with the first alignment feature 416a (for example, such that the alignment features 416 overlap), creating a first layer 422 (FIG. 12) and a second layer 424 (FIG. 12), with the reinforcing member 418 sandwiched between, as shown in FIG. 12.

As shown in FIG. 12, the layers 422, 424 and reinforcing member 418 can be secured together, for example, with a first suture or stitch line 426 (also called a primary stitch line). The first stitch line 426 can comprise plurality of stitches of a suture (for example, in-and-out stitches). The leaflet described previously can be a first leaflet 400a, and the commissure tab 412b of a second leaflet 400b can be folded in the same manner as the and placed against the folded commissure tab 412a of the first leaflet 400a within a commissure attachment member 428.

In some examples, the commissure attachment member 428 can be the same or similar to commissure attachment member 324 described previously. As shown in FIG. 12, when the commissure 402 is assembled, the side portions of the commissure attachment member 428 can extend laterally outwardly from the commissure adjacent a radially inner surface of a frame 434. Frame 434 can be similar to frame 152 described previously. In some examples, commissure attachment member 428 can be a have any of various shapes. For example, rectangular, diamond, circular, hexagonal, octagonal, oval, etc. In some examples, the outer perimeter of the commissure attachment member 428 when coupled to the commissure 402 can correspond to the shape of the outer perimeter of a cell of a frame of a prosthetic heart valve. In some examples, the commissure attachment member 428 can comprise a fabric, a flexible polymeric material, or a combination thereof. For example, the commissure attachment member can comprise polyethylene terephthalate (PET).

A second suture or stitch line 430a (also called a secondary stitch line) can extend diagonally through the first layer 422a of the first leaflet 400a, the second layer 424a, a portion of the second leaflet tab 412b, and the commissure attachment member 428. The secondary stitch line 430a can comprise plurality of stitches of a suture (for example, in-and-out stitches) and can be secured adjacent a radially outer facing surface 432 of the frame 434. The secondary stitch line 430 can partially overlap the primary stitch line 426, for example, the secondary stitch line 430a of the first leaflet 400a can share a radially inner origin point 436 with the primary stitch line 426a of the first leaflet 400a. In some examples, the secondary stitch line 430a can cross over and/or be sewn through the primary stitch line 426b of the second leaflet 400b.

As shown in FIG. 12, the secondary stitch lines 430a, 430b of the first and second leaflets can be arranged such that they cross over one another. Such a configuration advantageously helps retain the commissure 402 in a substantially perpendicular orientation relative to the frame 434 and centered within a cell of the frame 434. Referring to FIG. 14, the secondary stitch line 430 can be whip-stitched around one or more struts of the frame 434 to secure the commissure 402 to the frame and retain the commissure in the selected position. In some examples, the primary stitch line 426 can be whip-stitched around the frame. The origin points 436 of the primary and secondary suture lines 426, 430 can define a bending axis 438 (FIG. 12) extending through the origin points 436 of the two adjacent leaflets and around which the leaflets 400 bend upon opening (for example, during aortic systole).

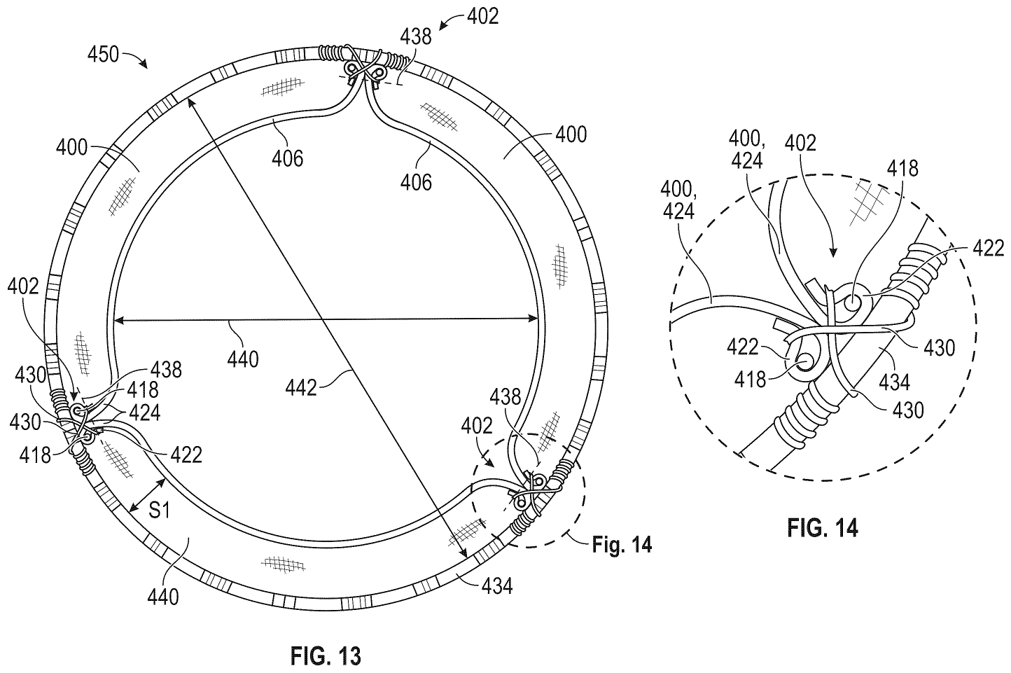

FIG. 13 illustrates a prosthetic valve 450 including a valvular structure comprising the leaflets 400 and commissures 402 described previously. As shown, the effective outflow orifice 440 (as determined by the open position of the leaflets 400; also referred to as the geometric orifice area (GOA)) has a smaller diameter than the outflow orifice 442 of the frame 434. The commissures 402 advantageously allow the bending axis 438 to be formed radially inwardly of the frame 434 to prevent the leaflets 400 from hitting against the frame, thus preventing and/or mitigating abrasion/damage and minimize the distance Si between the outflow edges 406 of the leaflets 400 and the frame 434 to maximize the effective outflow orifice 440. Such a configuration can advantageously improve the pressure gradient across the valve 450.

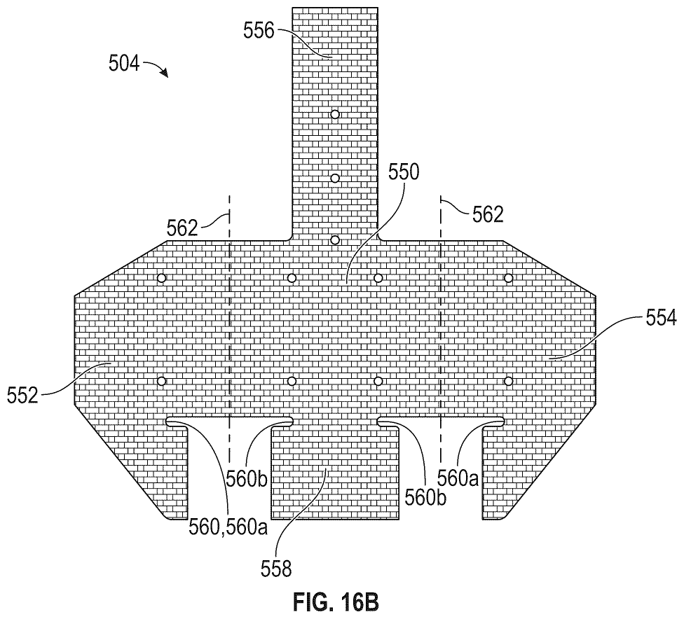

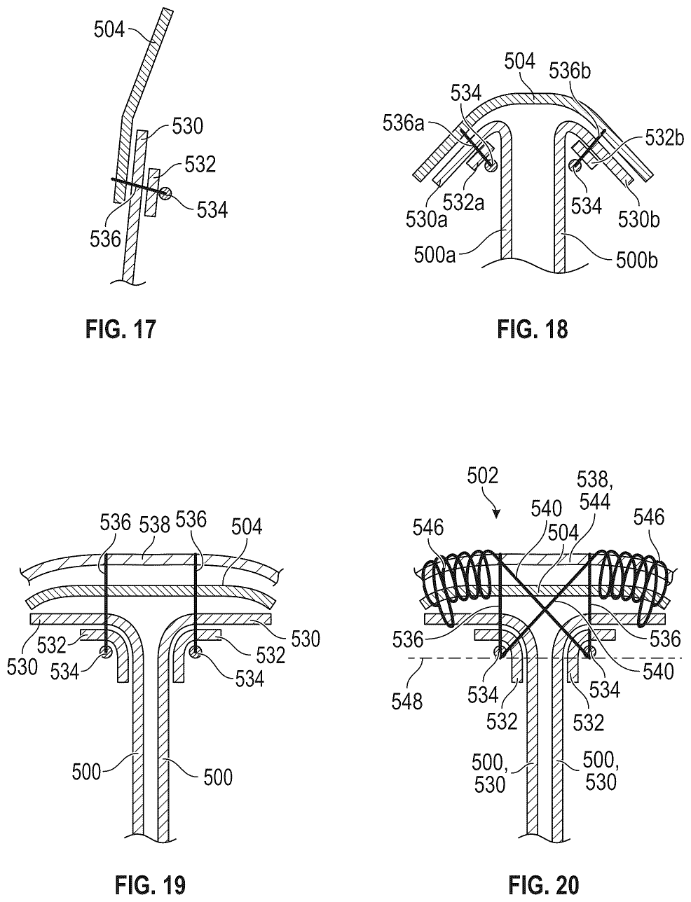

FIGS. 16A-23 illustrate an example of a leaflet 500 and a method for forming a commissure 502 (FIG. 20) from two leaflets 500 and a commissure attachment member 504 (FIG. 17), the commissure 502 being configured to help maximize the effective outflow orifice/GOA at the outflow end of a prosthetic valve, such as valve 150 described previously. The leaflet 500 can be similar to leaflet 300, except that the upper tab portions of leaflet 500 have a comparatively narrow shape.

In some examples, as shown in FIG. 16B the commissure attachment member 504 can be the same or similar to commissure attachment member 324 described previously, for example, having a main body portion 550, a first side portion 552, and an opposing second side portion 554. As shown, the outer peripheral edges of the side portions 552, 554 can be shaped to correspond to one half of a diamond-shaped cell of the frame to facilitate mounting of the commissure attachment member 504 to the struts of the frame. The commissure attachment member 504 can further comprise an optional upper extension 556 and an optional lower extension 558 that extend from the main body 550. In some examples, the upper extension 556 can comprise a radiopaque marker used to indicate the location of the commissure.

The commissure member 504 can further comprise one or more alignment notches 560 to facilitate folding of the first and side portions 552, 554 along vertical fold lines 562. Each side portion 552, 554 can be folded such that a first alignment notch 560a is disposed over a second alignment notch 560b, thereby forming first and second layers of material.

In some examples, commissure attachment member 504 can have any of various shapes. For example, rectangular, diamond, circular, hexagonal, octagonal, oval, etc. In some examples, the outer perimeter of the commissure attachment member 504 when coupled to the commissure 502 can correspond to the shape of the outer perimeter of a cell of a frame of a prosthetic heart valve. In some examples, the commissure attachment member 504 can comprise a fabric, a flexible polymeric material, or a combination thereof. For example, the commissure attachment member can comprise polyethylene terephthalate (PET).

As shown in FIG. 16A, the leaflet 500 comprises a main body 506, an upper edge portion 508 (also referred to as the coaptation edge), a lower edge portion 510 (also referred to as the cusp edge or scallop edge) terminating at openings/recesses/gaps 512 that extend laterally into the main body of the leaflet, and tabs 514 (also referred to as commissure tabs), which are spaced from the lower edge portion 510 by the gaps 512. In some examples, the tabs 514 can be formed integrally with the main body of the leaflet. The cusp edge 510 can be mounted to the frame 538 (FIG. 19), for example using a connecting skirt. The frame 538 can be similar to frame 152 described previously. In some examples, the leaflets 500 can be configured differently than the leaflet shown in FIG. 16A, such as having differently sized or shaped tabs 514 or differently sized or shaped gaps 512. As described further below and shown in FIGS. 17-20, the tabs 514 can be folded over on themselves and coupled to a commissure attachment member 504 to form commissures 502.

The tabs 514 can each comprise an upper tab portion 516 (also referred to as a ‘narrow tab’) and a lower tab portion 518. As shown in FIG. 16A, the upper tab portion 516 can be narrower than the lower tab portion 518 such that an outer edge 520 of the upper tab portion 516 is radially inset from an outer edge 522 of the lower tab portion 518, forming a stepped configuration. Such a configuration removes excess material from the commissure (for example, by allowing for fewer folded layers) while creating additional support (for example, multiple layers) at the bending axis (see for example, FIG. 20). In some examples, the upper tab portion 516 can have a width W₁ of about 1 mm. In some examples, the width W₁ can be any of various widths selected to maximize the GOA while preventing the main body of the leaflet from contacting the frame. For example, the width W₁ can be between about 0.1 mm and about 3 mm, about 0.5 mm and about 2 mm, about 0.7 mm and about 1.5 mm, etc.

In some examples, such as the illustrated example, the upper and lower tab portions 516, 518 and the cusp edge portion 510 can comprise one or more preformed openings or apertures 528 configured to facilitate sewing one or more components of the prosthetic valve to one another. In some examples, the openings may not be preformed in the leaflet 300.

As shown in FIG. 16A, the cusp edge portion 510 terminates at either end at the laterally projecting lower tab portions 518. The lower tab portions 518 can extend from the body 506 of the leaflet 500 such that an upper or outflow edge 524 of each lower tab portion 518 is positioned at an angle O relative to a longitudinal axis A of the leaflet 500. The angled tabs can advantageously improve the durability of the valvular structure by creating a buffer positioning the fold line of the leaflet radially inward of the frame such that the leaflet does not contact the frame during cycling (for example, during opening and closing of the valvular structure).

Referring now to FIGS. 17-20, to form each commissure 502, the upper tab portion 516 is folded along fold line 526 (FIG. 16A) against the lower tab portion 518, thus forming a first tab layer 530 (previously the lower tab portion 518) and a second tab layer 532 (previously the upper tab portion). In some examples, the upper tab portion 516 can be folded such that a line of apertures 528 of the upper tab portion 516 align with a line of apertures 528 of the lower tab portion 518. A reinforcing member 534 can be disposed on the second tab layer 532. The reinforcing member 418 can be, for example, a suture (for example, an ethibond suture), a strip of fabric (for example, PET), a braided cloth, a metallic braid, and/or a metal strip. The first and second tab layers 530, 532 and the reinforcing member 534 can be secured to the commissure attachment member 504 along a first stitch line 536 (also referred to as a primary stitch line) comprising a plurality of stitches of a suture (for example, in-and-out stitches).

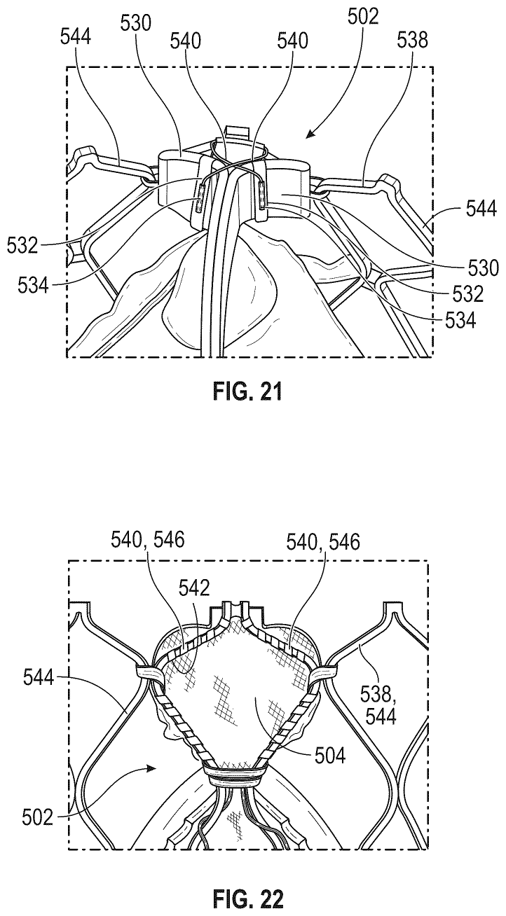

Referring to FIG. 18, the commissure tab 514b of a second leaflet 500b can be folded in the same manner and placed against the folded commissure tab 514a of the first leaflet 500a and secured to the commissure attachment member 504 against the radially inward facing surface using a primary stitch line 536b. As shown in FIG. 19, the primary stitch lines 536 can further secure the tab layers 530, 532 and commissure attachment member 504 to the frame 538 of a prosthetic valve, for example, within a selected cell 542 of the frame 538 (see for example, FIGS. 21-22).

Referring to FIG. 20, a secondary suture or stitch line 540 can extend diagonally through the reinforcing member 534, the first and second tab layers 530, 532, and the commissure attachment member 504 and can be secured to the frame 538 using, for example, a plurality of whip-stitches 546 extending around one or more struts 544 of the frame (see for example, FIG. 22). In some examples, the secondary stitch line 540 can be a continuation of the same suture from the primary stitch line 536. In some examples, such as shown in FIG. 20, one or more of the whip-stitches 546 can extend through the commissure attachment member 504 and/or the first tab layer 530 to retain the commissure attachment member 504 and/or the first tab layer 530 adjacent the frame 538.

The secondary stitch lines 540 of the two adjacent leaflets 500 can cross over one another to form an X-shape prior to being stitched to the frame 538. Such a configuration advantageously helps keep the commissure 502 perpendicular relative to the frame 538 and centered within the cell 542 of the frame.

The primary and secondary stitch lines can define a bending axis 548 (FIG. 20) around which the leaflets 500 can bend upon opening (for example, during aortic systole). As shown in FIG. 20, the bending axis 548 can extend laterally through both reinforcing members 534 of the commissure 502. Such a configuration allows the bending axis 548 to be formed radially inwardly of the frame 538, for example, to prevent the leaflets 500 from hitting against the frame 538, thus preventing and/or mitigating abrasion/damage, while also minimizing the distance between the outflow edges of the leaflets 500 and the frame 538 to maximize the effective outflow orifice/GOA at the outflow end portion of the prosthetic valve. Such a configuration can advantageously improve the pressure gradient across the valve.

FIGS. 24-29 illustrate an example of a leaflet 600, a commissure attachment member 604, and a method for forming a commissure 602 from two leaflets 600 and the commissure attachment member 604, the commissure 602 being configured to help maximize the effective outflow orifice/GOA at the outflow end of a prosthetic valve, such as valve 150 described previously. The leaflet 600 can be similar to leaflet 300 described previously, except that leaflet 600 does not include upper tab portions 318. The commissure attachment member 604 can be similar to commissure attachment member 324 described previously, except that commissure attachment member 604 has elongated side portions when compared to commissure attachment member 324.

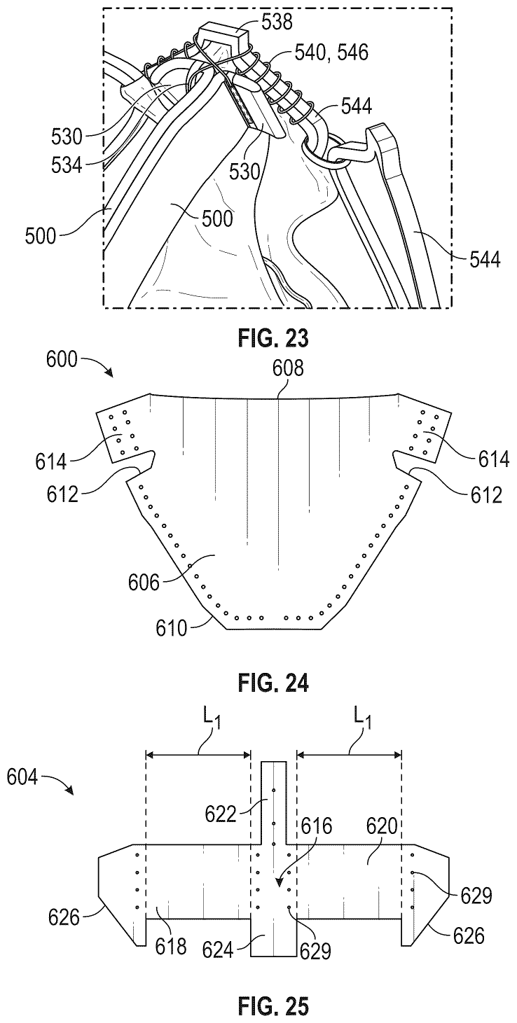

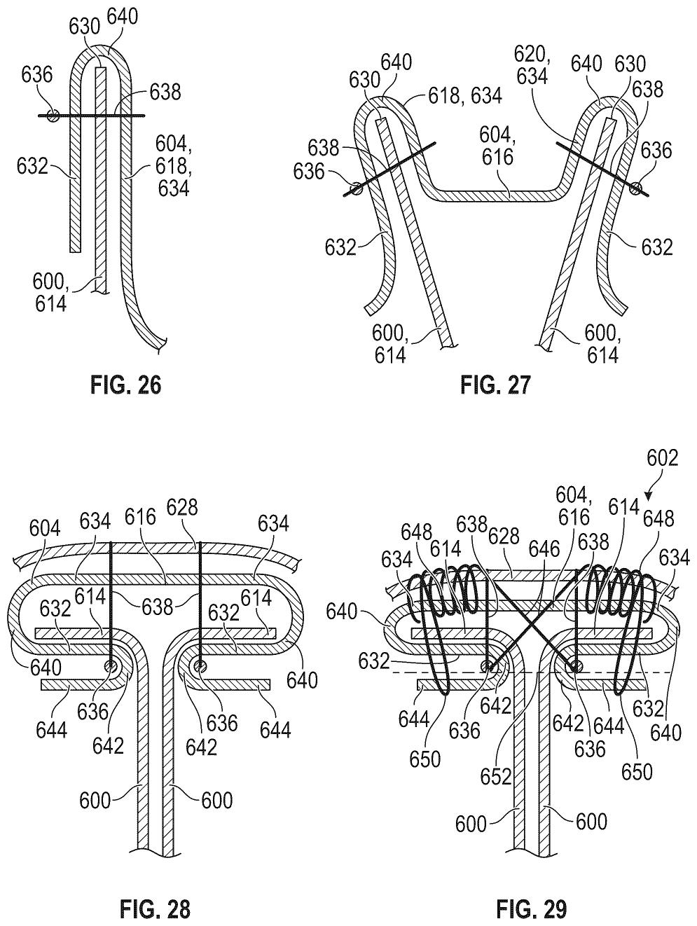

As shown in FIG. 24, the leaflet 600 comprises a main body 606, an upper edge portion 608 (also referred to as the coaptation edge), a lower edge portion 610 (also referred to as the cusp edge or scallop edge) terminating at openings/recesses/gaps 612 that extend laterally into the main body 606 of the leaflet, and tabs 614 (also referred to as commissure tabs), which are spaced from the lower edge portion by the gaps 612. The cusp edge 610 can be mounted to the frame of a prosthetic valve, for example using a connecting skirt. In alternate examples, the leaflets 600 can be configured differently than the leaflet shown in FIG. 23, such as having differently sized or shaped commissure tabs 614 or differently sized or shaped gaps 612. As described further below and shown in FIGS. 26-29, the tabs 614 can be folded over on themselves and coupled to a commissure attachment member 604 to form commissures 602.

Referring to FIG. 25, the commissure attachment member 604 can have a main body portion 616 and a first side portion 618 and an opposing second side portion 620 extending from the main body portion 616. The commissure attachment member 604 can further comprise an optional upper extension 622 and an optional lower extension 624 that extend from the main body 616. In some examples, the upper extension can comprise a radiopaque marker used to indicate the location of the commissure. An example of a commissure attachment member 604 in a flat and unfolded state (for example, prior to folding and commissure formation) is shown in FIG. 25. It should be noted that, in alternative examples, the attachment member 604 can have different configurations than shown in FIG. 25, such as not including the upper and/or lower extensions 622, 624, or having a different overall shape (for example a rectangular shape, a hexagonal shape, or the like). In some examples, the commissure attachment member 604 can comprise a fabric, a flexible polymeric material, or a combination thereof. For example, the commissure attachment member can comprise polyethylene terephthalate (PET).

Referring to FIG. 25, as shown, the outer peripheral edges 626 of the side portions 618, 620 can be shaped to correspond to one half of a diamond-shaped cell of the frame 628 (FIG. 28) to facilitate mounting of the commissure attachment member 604 to the frame 628. Frame 628 can be similar to frame 152 described previously. In some examples, such as the illustrated example, the commissure attachment member 604 can comprise one or more preformed openings 629 configured to facilitate sewing one or more components of the prosthetic valve to one another. In some examples, the openings may not be preformed in the commissure attachment member 604. In some examples, the commissure attachment member 604 can comprise a fabric, a flexible polymeric material, or a combination thereof.

As shown in FIG. 25, the first and second side portions 618 can have an elongated shape, for example, when compared to commissure attachment member 324. For example, the first and second side portions can each have a length L₁ between about 4 mm and about 5 mm, between about 3 mm and about 6 mm, or between about 2 mm and about 7 mm. In some examples, the length L₁ can be between about 4.5 mm and about 5 mm, or between about 4.6 mm and about 4.9 mm, between about 4.8 mm and about 4.9 mm.

Referring to FIGS. 26-29, to form each commissure 602, the first side portion 618 of the commissure attachment member 604 can be folded over a radially outer edge 630 of the leaflet tab 614, forming a U-shaped bend 640 defining two commissure member layers 632, 634 with the leaflet tab 614 sandwiched between them. As shown in FIG. 26, a reinforcing member 636 can be disposed on the first commissure member layer 632, and the first and second commissure member layers 632, 634, the leaflet tab 614, and the reinforcing member 636 can be secured together along a first stitch line 638 (also called the primary stitch line) comprising a plurality of stitches of a suture (for example, in-and-out stitches). The reinforcing member 636 can be, for example, a suture (for example, an ethibond suture), a strip of fabric (for example, PET), a braided cloth, a metallic braid, and/or a metal strip

Referring to FIG. 27, the commissure tab 614 of a second leaflet 600 can be secured to the second side portion 620 of the commissure attachment member 604 along with a reinforcing member 636 using a primary stitch line 638 in the same manner as described previously. As shown in FIG. 28, the primary stitch lines 638 can extend through a cell of the frame 628 to secure the commissure 602 to the frame 628. The first commissure layer 632 can be folded around the reinforcing member 636 to form a second U-shaped bend 642 defining a third layer 644.

As shown in FIG. 29, a secondary suture or stitch line 646 can extend diagonally through the reinforcing member 636, the first commissure member layer 632, the commissure tab 614, and the main body 616 of the commissure attachment member 604, and can be secured to the frame 628, for example using a whip-stitches 648 around one or more selected struts of the frame 628. As shown, the diagonally-extending portions of the secondary stitch lines 646 can cross over one another to form an X-shape prior to being stitched to the frame 628. Such a configuration advantageously helps keep the commissure 602 perpendicular relative to the frame 628 and centered within a cell of the frame. As seen in FIG. 29, one or more of the whip-stitches 648 can be elongated whip-stitches 650 that extend through the first, second, and third commissure member layers 632, 634, 644, and the leaflet tab 614 in order to secure the commissure 602 against the frame 628.

A bending axis 652 (FIG. 29) around which the leaflets bend upon opening (for example during aortic systole) can extend laterally through the reinforcing members 636 of the commissure 602. Such a configuration allows the bending axis 652 to be formed radially inwardly of the frame 628, for example, to prevent the working portion of the leaflets 600 (for example, the main body 606) from hitting against the frame 628, thus preventing and/or mitigating abrasion/damage to the leaflets. This configuration also advantageously minimizes the distance between the outflow edges of the leaflets 600 (in the open position) and the frame 628 to maximize the effective outflow orifice/GOA at the outflow end portion of the prosthetic valve. Such a configuration can advantageously improve the pressure gradient across the valve.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. An implantable prosthetic device, comprising:
   a radially expandable and compressible annular frame comprising an inflow end portion and an outflow end portion;
   a valvular structure comprising a plurality of leaflets arranged within the frame, each leaflet comprising a main body and one or more commissure tabs;
   a commissure comprising a commissure attachment member and the commissure tabs of two adjacent leaflets coupled to the commissure attachment member;
   wherein the commissure is secured to the frame via first and second sutures, each suture extending through a respective commissure tab, through the commissure attachment member, crossing over the other suture and being secured to the frame; and
   wherein the commissure defines a bending axis disposed radially inwardly of the frame such that the main body of each leaflet is prevented from contacting the frame when the valvular structure is in an open configuration.
Example 2. The prosthetic device of any example herein, particularly example 1, wherein each commissure further comprises a reinforcing member disposed on the commissure tab.
Example 3. The prosthetic device of any example herein, particularly example 2, wherein the reinforcing member comprises an ethibond suture.
Example 4. The prosthetic device of any example herein, particularly example 2, wherein the reinforcing member comprises a strip of PET fabric.
Example 5. The prosthetic device of any example herein, particularly any one of examples 2-4, wherein the reinforcing member is a first reinforcing member and wherein each commissure further comprises a second reinforcing member.
Example 6. The prosthetic device of any example herein, particularly example 5, wherein the commissure comprises third and fourth sutures and wherein each of the third and fourth sutures extend through a respective reinforcing member, a respective commissure tab, and the commissure attachment member.
Example 7. The prosthetic device of any example herein, particularly any one of examples 1-6, wherein the commissure attachment member comprises first and second side portions extending from a main portion, and wherein the commissure attachment member is folded such that the first and second side portions each have a first layer and a second layer.
Example 8. The prosthetic device of any example herein, particularly any one of examples 1-7, wherein each commissure tab comprises an upper tab portion and a lower tab portion, and wherein the upper tab portion of each tab is folded toward the inflow end of the frame to form first and second tab layers.
Example 9. The prosthetic device of any example herein, particularly example 8, wherein an outer edge of the lower tab portion extends laterally past an outer edge of the upper tab portion.
Example 10. The prosthetic device of any example herein, particularly any one of examples 8-9, wherein an outflow edge of the lower tab is disposed at an angle relative to a longitudinal axis of the leaflet, and wherein the angle is a non-90 degree angle.
Example 11. The prosthetic device of any example herein, particularly any one of examples 8-10, wherein a radially inner edge portion of the first tab layer can wrap around a portion of the commissure attachment member such that the main body of the leaflet contacts the first tab layer when the valvular structure is in the open configuration.
Example 12. The prosthetic device of any example herein, particularly any one of examples 1-11, wherein the commissure tabs are formed integrally with the main body of a respective leaflet.
Example 13. The prosthetic device of any example herein, particularly any one of examples 1-12, wherein the valvular structure comprises three leaflets, each coupled one or more adjacent leaflets at a respective commissure.
Example 14. A method of assembling a valvular structure, comprising:
   folding an integral upper tab portion of a first leaflet onto an integral lower tab portion of the first leaflet to form first and second tab layers;
   disposing a first reinforcing member on the second tab layer of the first leaflet;
   disposing the first and second tab layers on a first side portion of a commissure attachment member;
   coupling the first reinforcing member, the first tab layer, the second tab layer, and the first side portion of the commissure attachment member to one another using one or more sutures;
   folding an integral upper tab portion of a second leaflet onto an integral lower tab portion of the first leaflet to form first and second tab layers;
   disposing a second reinforcing member on the second tab layer of the second leaflet;
   disposing the first and second tab layers on a second side portion of the commissure attachment member; and
   coupling the second reinforcing member, the first tab layer and the second tab layer of the second leaflet, and the second side portion of the commissure attachment member to one another using one or more sutures to form a commissure.
Example 15. The method of any example herein, particularly example 14, further comprising coupling the commissure to a radially expandable and compressible annular frame.
Example 16. The method of any example herein, particularly any one of examples 14-15, further comprising:
   prior to disposing the first and second tab layers on the first side portion of the commissure attachment member, folding the commissure attachment member such that each of the first side portion and the second side portion comprise first and second commissure member layers.
Example 17. An implantable prosthetic device, comprising:
   a frame comprising an inflow end portion and an outflow end portion, the frame being radially collapsible to a collapsed configuration and radially expandable to an expanded configuration;
   a valvular structure comprising a plurality of leaflets arranged within the frame, each leaflet comprising a main body and a pair of opposing commissure tabs extending from opposite sides of the main body, each commissure tab having a lower tab portion and an upper tab portion;
   a commissure attachment member comprising a main body portion and first and second side portions;
   a commissure comprising the commissure tabs of respective first and second adjacent leaflets coupled to the commissure attachment member, the upper tab portion of each commissure tab being folded toward the inflow end of the frame to form first and second tab layers;
   wherein a first suture couples the first and second tab layers of the first leaflet to the first side portion of the commissure attachment member and a second suture couples the first and second tab layers of the second leaflet to the second side portion;
   wherein a third suture extends through the first side portion, the first tab layer of the first leaflet, the main body portion of the commissure attachment member, and is secured to the frame, and a fourth suture extends through the second side portion, the first tab layer of the second leaflet, the main body portion of the commissure attachment member and is secured to the frame;
   wherein the third and fourth sutures cross over one another adjacent the frame; and
   wherein the commissure defines a bending axis disposed radially inwardly of the frame such that the main body of the leaflet is prevented from contacting the frame when the valvular structure is in an open configuration.
Example 18. The prosthetic device of any example herein, particularly example 17, wherein each commissure further comprises a first reinforcing member disposed on the second tab layer of the first leaflet, and a second reinforcing member disposed on the second tab layer of the second leaflet.
Example 19. The prosthetic device of any example herein, particularly example 18, wherein the first and second sutures extend through the first and second reinforcing members, respectively.
Example 20. The prosthetic device of any example herein, particularly example 19, wherein the third and fourth sutures extend through the first and second reinforcing members, respectively.
Example 21. The prosthetic device of any example herein, particularly example 18, wherein the first and second reinforcing members comprise ethibond sutures.
Example 22. The prosthetic device of any example herein, particularly example 18, wherein the first and second reinforcing members comprise strips of PET fabric.
Example 23. The prosthetic device of any example herein, particularly any one of examples 17-22, wherein the commissure attachment member is folded such that the first and second side portions each have a first layer and a second layer.
Example 24. The prosthetic device of any example herein, particularly example 23, wherein the first suture extends through the first and second layers of the first side portion of the commissure attachment member and wherein the second suture extends through the first and second layers of the second side portion of the commissure attachment member.
Example 25. The prosthetic device of any example herein, particularly any one of examples 17-24, wherein an outer edge of the lower tab portion of a respective commissure tab extends laterally past an outer edge of the upper tab portion of the respective commissure tab.
Example 26. The prosthetic device of any example herein, particularly any one of examples 17-25, wherein an outflow edge of the lower tab is disposed at an angle relative to a longitudinal axis of the leaflet, and wherein the angle is a non-90 degree angle.
Example 27. The prosthetic device of any example herein, particularly any one of examples 17-26, wherein a radially inner edge portion of the first tab layer can wrap around a portion of the commissure attachment member such that the main body of the leaflet contacts the first tab layer when the valvular structure is in the open configuration.
Example 28. The prosthetic device of any example herein, particularly example 27, wherein each radially inner edge portion of each first tab layer wraps around the first and second layers of a respective side portion of the commissure attachment member.
Example 29. The prosthetic device of any example herein, particularly any one of examples 17-28, wherein the third and fourth sutures cross over one another at a location radially inward of the frame.
Example 30. The prosthetic device of any example herein, particularly any one of examples 17-29, wherein the third and fourth sutures are secured to the frame using one or more whip stitches.
Example 31. An implantable prosthetic device, comprising:
   a radially expandable and compressible annular frame comprising an inflow end portion and an outflow end portion;
   a valvular structure comprising a plurality of leaflets arranged within the frame, each leaflet comprising a main body and one or more commissure tabs, an outer portion of each commissure tab being folded toward a longitudinal axis of the leaflet to form first and second tab layers;
   a commissure comprising a commissure attachment member and the commissure tabs of two adjacent leaflets coupled to the commissure attachment member;
   wherein the commissure is secured to the frame via first and second sutures, each suture extending through the first tab layer, the second tab layer, the commissure attachment member, crossing over the other suture and being secured to the frame; and
   wherein the commissure defines a bending axis disposed radially inwardly of the frame such that the main body of each leaflet is prevented from contacting the frame when the valvular structure is in an open configuration.
Example 32. The prosthetic device of any example herein, particularly example 31, wherein a reinforcing member is disposed between the first and second tab layers of each commissure tab.
Example 33. The prosthetic device of any example herein, particularly example 31, wherein the commissure further comprises a third suture extending through the first tab layer, reinforcing member, and second tab layer of a respective first leaflet, and a fourth suture extending through the first tab layer, reinforcing member, and second tab layer of a respective second leaflet.
Example 34. The prosthetic device of any example herein, particularly any one of examples 31-33, wherein each commissure tab comprises first and second alignment features, and wherein when the outer portion is folded toward the longitudinal axis the first alignment feature aligns with the second alignment feature.
Example 35. The prosthetic device of any example herein, particularly example 34, wherein the alignment features comprise notches disposed on an inflow edge portion of the tab.
Example 36. The prosthetic device of any example herein, particularly example 35, wherein the notches have a triangular shape.
Example 37. The prosthetic device of any example herein, particularly any one of examples 31-36, wherein each leaflet comprises two commissure tabs and the two commissure tabs are formed integrally with the main body of the leaflet.
Example 38. The prosthetic device of any example herein, particularly any one of examples 31-37, wherein the first and second sutures cross over one another at a location radially inward of the frame.
Example 39. An implantable prosthetic device, comprising:
   a radially expandable and compressible frame comprising an inflow end portion and an outflow end portion;
   a valvular structure comprising a plurality of leaflets arranged within the frame, each leaflet comprising a main body and one or more commissure tabs, each commissure tab comprising a first alignment feature on a laterally outer tab portion and a second alignment feature on a laterally inner tab portion, the laterally outer portion of each commissure tab being folded over the laterally inner portion such that the first alignment feature is disposed on the second alignment feature to form first and second tab layers;
   a commissure comprising a commissure attachment member and the commissure tabs of a first leaflet and an adjacent second leaflet coupled to the commissure attachment member, the commissure comprising a first suture extending through the first and second tab layers of the first leaflet and a second suture extending through the first and second tab layers of the second leaflet;
   wherein the commissure is secured to the frame via third and fourth sutures, the third suture extending through the first and second tab layers of the first leaflet, the commissure attachment member, crossing over the fourth suture, and being secured to the frame at a first location, and the fourth suture extending through the first and second tab layers of the second leaflet, the commissure attachment member, crossing over the third suture, and being secured to the frame at a second location; and
   wherein the commissure defines a bending axis disposed radially inwardly of the frame such that the main body of each leaflet is prevented from contacting the frame when the valvular structure is in an open configuration.
Example 40. The prosthetic device of any example herein, particularly example 39, further comprising a first reinforcing member disposed between the first and second tab layers of the first leaflet and a second reinforcing member disposed between the first and second tab layers of the second leaflet.
Example 41. The prosthetic device of any example herein, particularly example 40, wherein the first suture extends through the first reinforcing member and the second suture extends through the second reinforcing member.
Example 42. The prosthetic device of any example herein, particularly any one of examples 39-41, wherein the first suture and the third suture share a first origin point, and wherein the second suture and the fourth suture share a second origin point.
Example 43. A method of assembling a valvular structure, comprising:
   disposing a first reinforcing member on a laterally inner tab portion of a commissure tab of a first leaflet such that the first reinforcing member is aligned with a first alignment feature of the laterally inner tab portion;
   folding a laterally outer tab portion laterally over the first reinforcing member such that a second alignment feature disposed on the outer tab portion of the commissure tab of the first leaflet aligns with the first alignment feature, and such that the commissure tab forms first and second tab layers;
   coupling the first and second tab layers and the first reinforcing member using one or more first sutures;
   disposing the first and second tab layers and the first reinforcing member on a first side portion of a commissure attachment member;
   disposing a second reinforcing member on a laterally inner tab portion of a commissure tab of a second leaflet such that the second reinforcing member is aligned with a first alignment feature of the laterally inner tab portion;
   folding a laterally outer tab portion of the second leaflet laterally over the second reinforcing member such that a second alignment feature disposed on the outer tab portion of the commissure tab of the second leaflet aligns with the first alignment feature, and such that the commissure tab forms first and second tab layers;
   coupling the first and second tab layers and the second reinforcing member using one or more second sutures;
   disposing the first and second tab layers and the second reinforcing member on a second side portion of the commissure attachment member to form a commissure; and
   coupling the commissure to a radially expandable and compressible annular frame via one or more third sutures extending diagonally through the first and second tab layers of the first leaflet and one or more fourth sutures extending diagonally through the first and second tab layers of the second leaflet, the third and fourth sutures crossing over one another at a location radially inside the frame.
Example 44. An implantable prosthetic device, comprising:
   a radially expandable and compressible annular frame comprising an inflow end portion and an outflow end portion;
   a valvular structure comprising a plurality of leaflets arranged within the frame, each leaflet comprising a main body and one or more commissure tabs, each commissure tab having an upper tab portion and a lower tab portion, the upper tab portion being folded toward an inflow end portion of the leaflet to form first and second tab layers;
   one or more reinforcing members each disposed on a respective second tab layer of a commissure tab;
   a commissure comprising a commissure attachment member and the commissure tabs of two adjacent leaflets coupled to the commissure attachment member;
   wherein the commissure is secured to the frame via first and second sutures, each suture extending radially through a respective reinforcing member and the first and second tab layers of the two adjacent leaflets and being secured to the frame; and
   wherein the commissure defines a bending axis disposed radially inwardly of the frame such that the main body of each leaflet is prevented from contacting the frame when the valvular structure is in an open configuration.
Example 45. The prosthetic device of any example herein, particularly example 44, wherein the upper tab portion has a width of 1 mm.
Example 46. The prosthetic device of any example herein, particularly any one of examples 44-45, further comprising a third suture extending diagonally through the commissure in a first direction and a fourth suture extending diagonally through the commissure in a second direction such that the third and fourth sutures cross over one another in an X shape.
Example 47. The prosthetic device of any example herein, particularly example 46, wherein the third and fourth sutures extend around respective struts of the frame in a whip-stitch pattern.
Example 48. The prosthetic device of any example herein, particularly example 47, wherein one or more of the whip stitches extend through the commissure attachment member.
Example 49. The prosthetic device of any example herein, particularly any one of examples 47-48, wherein one or more of the whip stitches extend through the first tab layer of at least one of the adjacent leaflets.
Example 50. The prosthetic device of any example herein, particularly any one of examples 44-49, wherein the bending axis extends laterally through the reinforcing members.
Example 51. The prosthetic device of any example herein, particularly any one of examples 44-50, wherein one or more reinforcing members comprise ethibond sutures.
Example 52. The prosthetic device of any example herein, particularly example 51, wherein the one or more reinforcing members comprise strips of PET fabric.
Example 53. The prosthetic device of any example herein, particularly any one of examples 44-52, wherein an outer edge of the lower tab portion extends laterally past an outer edge of the upper tab portion.
Example 54. The prosthetic device of any example herein, particularly any one of examples 44-53, wherein an outflow edge of the lower tab is disposed at an angle relative to a longitudinal axis of the leaflet, and wherein the angle is a non-90 degree angle.
Example 55. The prosthetic device of any example herein, particularly any one of examples 44-54, wherein the commissure tabs are formed integrally with the main body of a respective leaflet.
Example 56. A method of assembling a valvular structure, comprising:
   folding an integral upper tab portion of a first leaflet onto an integral lower tab portion of the first leaflet to form first and second tab layers;
   disposing a first reinforcing member on the second tab layer of the first leaflet;
   disposing the first and second tab layers on a first side portion of a commissure attachment member;
   coupling the first reinforcing member, the first tab layer, the second tab layer, and the first side portion of the commissure attachment member to one another using one or more sutures;
   folding an integral upper tab portion of a second leaflet onto an integral lower tab portion of the second leaflet to form first and second tab layers;
   disposing a second reinforcing member on the second tab layer of the second leaflet;
   disposing the first and second tab layers on a second side portion of the commissure attachment member;
   coupling the second reinforcing member, the first tab layer and the second tab layer of the second leaflet, and the second side portion of the commissure attachment member to one another using one or more sutures to form a commissure; and
   coupling the commissure to a radially expandable and compressible annular frame via one or more third sutures extending diagonally through the first reinforcing member and the first and second tab layers of the first leaflet and one or more fourth sutures extending diagonally through the second reinforcing member and the first and second tab layers of the second leaflet, the third and fourth sutures crossing over one another at a location radially inside the frame.
Example 57. The method of any example herein, particularly example 56, further comprising:
   securing the one or more third and fourth sutures to selected struts of the frame using a whip-stitch pattern.
Example 58. The method of any example herein, particularly example 57, further comprising:
   stitching one or more of the whip stitches through the commissure attachment member.
Example 59. The method of any example herein, particularly any one of examples 57-58, further comprising:
   stitching one or more of the whip stitches through the first tab layer of at least one of the first leaflet and the second leaflet.
Example 60. An implantable prosthetic device, comprising:
   a radially expandable and compressible annular frame comprising an inflow end portion and an outflow end portion;
   a valvular structure comprising a plurality of leaflets arranged within the frame, each leaflet comprising a main body and one or more commissure tabs;
   a commissure attachment member comprising a main body, a first side portion, and a second side portion;
   a commissure comprising the commissure attachment member and the commissure tabs of first and second adjacent leaflets coupled to the commissure attachment member, the first side portion of the commissure attachment member being folded over a radially outer edge of a first commissure tab of the first leaflet to form first and second commissure member layers, and the second side portion of the commissure attachment member being folded over a radially outer edge of a second commissure tab of the second leaflet to form first and second commissure member layers;
   wherein the commissure is secured to the frame via first and second sutures, the first suture extending radially through the first and second commissure member layers of the first side portion and the first commissure tab, the second suture extending radially through the first and second commissure member layers of the second side portion and the second commissure tab; and
   wherein the commissure is further secured to the frame via a third suture extending diagonally through the commissure in a first direction and a fourth suture extending diagonally through the commissure in a second direction such that the third and fourth sutures cross over one another in an X shape, the third and fourth sutures extending around respective struts of the frame in a plurality of whip stitches.
Example 61. The prosthetic device of any example herein, particularly example 60, further comprising first and second reinforcing members, the first reinforcing member being disposed on the first commissure member layer of the first commissure tab and the second reinforcing member being disposed on the first commissure member layer of the second commissure tab.
Example 62. The prosthetic device of any example herein, particularly any one of examples 60-61, wherein one or more of the whip stitches extend through the second commissure member layer of the first side portion and the second side portion.
Example 63. The prosthetic device of any example herein, particularly any one of examples 60-62, wherein the first commissure member layer is folded laterally to form a third commissure member layer disposed radially inwardly of the first commissure member layer.
Example 64. The prosthetic device of any example herein, particularly example 63, wherein one or more of the whip stitches extend through the third commissure member layer of the first leaflet and the second leaflet.
Example 65. The prosthetic device of any example herein, particularly any one of examples 60-64, wherein the first and second side portions of the commissure attachment member have an elongated shape.
Example 66. A method of assembling a valvular structure, comprising:
   folding a first side portion of a commissure attachment member laterally toward a main body of the commissure attachment member to form a first commissure member layer and a second commissure member layer;
   folding a second side portion of the commissure attachment member laterally toward the main body to form a first commissure member layer and a second commissure member layer;
   disposing a radially outer edge of a first commissure tab of a first leaflet between the first and second commissure member layers of the first side portion;
   disposing a radially outer edge of a second commissure tab of a second leaflet between the first and second commissure member layers of the second side portion;
   disposing a first reinforcing member on the first commissure member layer of the first side portion and a second reinforcing member on the first commissure member layer of the second side portion;
   coupling the first reinforcing member, the first and second commissure member layers of the first side portion, and the first commissure tab one another using one or more first sutures;
   coupling the second reinforcing member, the first and second commissure member layers of the second side portion, and the second commissure tab one another using one or more second sutures to form a commissure;
   coupling the commissure to a radially expandable and compressible annular frame via the first and second sutures;
   sewing one or more third sutures diagonally through the commissure in a first direction and one or more fourth sutures diagonally through the commissure in a second direction such that the third and fourth sutures cross over one another in an X shape; and
   securing the one or more third and fourth sutures to selected struts of the frame using a whip-stitch pattern.
Example 67. The method of any example herein, particularly example 66, further comprising:
   stitching one or more of the whip stitches through the commissure attachment member.
Example 68. The method of any example herein, particularly any one of examples 66-67, further comprising:
   folding each first commissure member layer laterally to form a third commissure member layer; and
   stitching one or more of the whip stitches through the third commissure member layer.
Example 69. An implantable prosthetic device according to any one of examples 1-68, wherein the device is sterilized.
Example 70. A method, comprising:
   sterilizing an implantable prosthetic device as described in any one of examples 1-68.

The features described herein with regard to any example can be combined with other features described in any one or more of the examples, unless otherwise stated. For example, any one or more of the features of one leaflet or commissure attachment member can be combined with any one or more features of another leaflet or commissure attachment member. As another example, any one or more features of one commissure can be combined with any one or more features of another commissure.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. An implantable prosthetic device, comprising:
a radially expandable and compressible annular frame comprising an inflow end portion and an outflow end portion;
a valvular structure comprising a plurality of leaflets arranged within the frame, each leaflet comprising a main body and one or more commissure tabs;
a commissure comprising a commissure attachment member and the commissure tabs of two adjacent leaflets coupled to the commissure attachment member;
wherein the commissure is secured to the frame via first and second sutures, each suture extending through a respective commissure tab, through the commissure attachment member, crossing over the other suture and being secured to the frame; and
wherein the commissure defines a bending axis disposed radially inwardly of the frame such that the main body of each leaflet is prevented from contacting the frame when the valvular structure is in an open configuration.

2. The prosthetic device of claim 1, wherein each commissure further comprises a reinforcing member disposed on the commissure tab.

3. The prosthetic device of claim 2, wherein the reinforcing member comprises an ethibond suture.

4. The prosthetic device of claim 2, wherein the reinforcing member comprises a strip of PET fabric.

5. The prosthetic device of claim 2, wherein the reinforcing member is a first reinforcing member and wherein each commissure further comprises a second reinforcing member.

6. The prosthetic device of claim 5, wherein the commissure comprises third and fourth sutures and wherein each of the third and fourth sutures extend through a respective reinforcing member, a respective commissure tab, and the commissure attachment member.

7. The prosthetic device of claim 1, wherein the commissure attachment member comprises first and second side portions extending from a main portion, and wherein the commissure attachment member is folded such that the first and second side portions each have a first layer and a second layer.

8. The prosthetic device of claim 1, wherein each commissure tab comprises an upper tab portion and a lower tab portion, and wherein the upper tab portion of each tab is folded toward the inflow end of the frame to form first and second tab layers.

9. The prosthetic device of claim 8, wherein an outer edge of the lower tab portion extends laterally past an outer edge of the upper tab portion.

10. The prosthetic device of claim 8, wherein an outflow edge of the lower tab is disposed at an angle relative to a longitudinal axis of the leaflet, and wherein the angle is a non-90 degree angle.

11. The prosthetic device of claim 8, wherein a radially inner edge portion of the first tab layer can wrap around a portion of the commissure attachment member such that the main body of the leaflet contacts the first tab layer when the valvular structure is in the open configuration.

12. The prosthetic device of claim 1, wherein the commissure tabs are formed integrally with the main body of a respective leaflet.

13. The prosthetic device of claim 1, wherein the valvular structure comprises three leaflets, each coupled one or more adjacent leaflets at a respective commissure.

14. A method of assembling a valvular structure, comprising:
folding an integral upper tab portion of a first leaflet onto an integral lower tab portion of the first leaflet to form first and second tab layers;
disposing a first reinforcing member on the second tab layer of the first leaflet;
disposing the first and second tab layers on a first side portion of a commissure attachment member;
coupling the first reinforcing member, the first tab layer, the second tab layer, and the first side portion of the commissure attachment member to one another using one or more sutures;
folding an integral upper tab portion of a second leaflet onto an integral lower tab portion of the first leaflet to form first and second tab layers;
disposing a second reinforcing member on the second tab layer of the second leaflet;
disposing the first and second tab layers on a second side portion of the commissure attachment member; and
coupling the second reinforcing member, the first tab layer and the second tab layer of the second leaflet, and the second side portion of the commissure attachment member to one another using one or more sutures to form a commissure.

15. The method of claim 14, further comprising coupling the commissure to a radially expandable and compressible annular frame.

16. The method of claim 14, further comprising:
prior to disposing the first and second tab layers on the first side portion of the commissure attachment member, folding the commissure attachment member such that each of the first side portion and the second side portion comprise first and second commissure member layers.

17. An implantable prosthetic device, comprising:
a frame comprising an inflow end portion and an outflow end portion, the frame being radially collapsible to a collapsed configuration and radially expandable to an expanded configuration;
a valvular structure comprising a plurality of leaflets arranged within the frame, each leaflet comprising a main body and a pair of opposing commissure tabs extending from opposite sides of the main body, each commissure tab having a lower tab portion and an upper tab portion;
a commissure attachment member comprising a main body portion and first and second side portions;
a commissure comprising the commissure tabs of respective first and second adjacent leaflets coupled to the commissure attachment member, the upper tab portion of each commissure tab being folded toward the inflow end of the frame to form first and second tab layers;
wherein a first suture couples the first and second tab layers of the first leaflet to the first side portion of the commissure attachment member and a second suture couples the first and second tab layers of the second leaflet to the second side portion;
wherein a third suture extends through the first side portion, the first tab layer of the first leaflet, the main body portion of the commissure attachment member, and is secured to the frame, and a fourth suture extends through the second side portion, the first tab layer of the second leaflet, the main body portion of the commissure attachment member and is secured to the frame;
wherein the third and fourth sutures cross over one another adjacent the frame; and
wherein the commissure defines a bending axis disposed radially inwardly of the frame such that the main body of the leaflet is prevented from contacting the frame when the valvular structure is in an open configuration.

18. The prosthetic device of claim 17, wherein each commissure further comprises a first reinforcing member disposed on the second tab layer of the first leaflet, and a second reinforcing member disposed on the second tab layer of the second leaflet.

19. The prosthetic device of claim 18, wherein the first and second sutures extend through the first and second reinforcing members, respectively.

20. The prosthetic device of claim 19, wherein the third and fourth sutures extend through the first and second reinforcing members, respectively.
